(19) United States
(12) Patent Application Publication
(10) Pub. No.: US 2026/0014103 A1
(43) Pub. Date: Jan. 15, 2026

(54) COMBINATION REMEDY

(71) Applicant: SYMRISE AG, Holzminden (DE)

(72) Inventors: Jakob Ley, Holzminden (DE); Veronika Somoza, Weidling (AT); Verena Stöger, Vienna (AT); Joachim Hans, Holzminden (DE)

(21) Appl. No.: 19/330,192

(22) Filed: Sep. 16, 2025

Related U.S. Application Data
(63) Continuation of application No. 17/423,400.

(51) Int. Cl. A61K 31/165; A23L 33/175; A23L 33/18; A61K 31/198; A61K 38/01; A61K 45/06

(52) U.S. Cl. CPC A61K 31/165; A23L 33/175; A23L 33/18; A61K 31/198; A61K 38/011; A61K 38/012; A61K 45/06

(57) ABSTRACT

The present invention primarily relates to a combination agent for reducing appetite and/or for providing a feeling of satiety and/or for reducing energy intake and/or for reducing body weight. In addition, the present invention relates to a medical use of the combination agent, in particular for the treatment of overweight and obesity and for the prevention of cardiovascular disease, stroke, diabetes, and joint damage and wear of the spine, and to a non-therapeutic use of the combination agent. In particular, the focus of the present invention is on the use of the combination agent to provide a feeling of satiety and/or to reduce energy intake and/or to reduce body weight. Finally, the present invention relates to the use of the combination agent for the preparation of an orally consumable preparation or food ingredient and an orally consumable preparation or food ingredient prepared therefrom.

### FIELD OF INVENTION

The present invention primarily relates to a combination agent for reducing appetite and/or for providing a feeling of satiety and/or for reducing energy intake and/or for reducing body weight. In addition, the present invention relates to a medical use of the combination agent, in particular for the treatment of overweight and obesity and for the prevention of cardiovascular disease, stroke, diabetes, and joint damage and wear of the spine, and to a non-therapeutic use of the combination agent. In particular, the focus of the present invention is on the use of the combination agent to provide a feeling of satiety and/or to reduce energy intake and/or to reduce body weight. Finally, the present invention relates to the use of the combination agent for the preparation of an orally consumable preparation or food ingredient and an orally consumable preparation or food ingredient prepared therefrom.

### STATE OF THE ART

The mechanism of satiety is a very complex physical process involving numerous factors, not all of which have yet been researched. Basically, hunger and satiety serve to regulate human food intake and to ensure adequate supply of the organism.

It is known that hunger and satiety are mediated by a specific region in the brain, the hypothalamus. Two centers operate here, hunger center and satiety center. These centers regulate a release of hormones into the blood that induce either food intake or eating cessation: The hunger center induces the release of the “hunger hormones” neuropeptide Y, agouti related peptide (AGRP), and melanin concentrating hormone (MCH), as well as cannabis-like hormones (endocannaboids). The satiety center works primarily with the “satiety hormones” propiomelanocortin (alpha-MSH), CART, and serotonin. How strongly the hunger center and the satiety center work depends on signals from the body: a falling blood glucose level signals hunger, while insulin, which is increasingly secreted when blood glucose levels are high, activates the satiety center.

Signals from the gastrointestinal tract also play a role: afferent signals are transmitted to the central nervous system by the stretch receptors of the stomach and intestine after the stomach is filled during eating. In addition, certain hormones in the stomach and intestine (such as leptin, cholecystokinin, or the glucagon like peptide 1) are released and stimulate the satiety center. Furthermore, recent studies show that an increased level of peripherally occurring serotonin, which is secreted by enterochromaffin cells in the intestine after stimulation, is also associated with an enhanced feeling of satiety (Hochkogler, Christina M. et al., The capsaicin analog nonivamide decreases total energy intake from a standardized breakfast and enhances plasma serotonin levels in moderately overweight men after administered in an oral glucose tolerance test: A randomized, crossover trial, Molecular nutrition & food research, 58.6 (2014): 1282-1290). Conversely, lower plasma serotonin levels were associated with increased appetite (Hochkogler, Christina M. et al., Appetite-inducing effects of homoeriodictyol: Two randomized, cross-over interventions, Molecular nutrition & food research, 61.12 (2017): 1700459).

The frequent occurrence of persistent obesity caused by a lack of exercise and/or excessive food intake and/or disorders of the satiety mechanism, such as obesity, a nutritional and metabolic disease, and eating disorders, such as binge eating (food cravings), can lead to secondary diseases such as insulin resistance, lipid metabolism disorders, high blood pressure, type II diabetes, arteriosclerosis, heart or cerebral infarction, and ultimately premature death. In particular, a high content of easily metabolized carbohydrates, proteins and especially fats in the diet leads to the formation of fat deposits and can ultimately contribute greatly to the problems mentioned above.

To counteract increased intake of calorically relevant food components, there has long been a desire to provide oral preparations that can reduce the feeling of hunger and natural appetite and/or increase the feeling of satiety accordingly.

In order to limit the intake of food components that are often highly preferred hedonically, especially fats and sweet carbohydrates (sugars), their contents are greatly reduced in calorie-reduced foods, the so-called “light products”, and these are often replaced by substitutes (thickeners for fats, non-caloric sweeteners instead of sugars).

However, when consuming “light products”, a product which, due to clever formulation, has a comparable hedonic value to the high-energy original product, may be consumed in larger quantities and then, in the worst case, there may even be an increased intake of calorically relevant food components. In such cases, the goal of lowering the amount of calories consumed is missed.

In the treatment of overweight and obesity, various methods are used to limit the food intake of those affected. Dietary fibers with a high water-binding capacity, such as glucomannans (konjac) or psyllium husks, have a moderate effect.

In addition, there are various anorectic agents (appetite suppressants) on the market. Anorectics are drugs with an appetite-inhibiting effect. This effect is based on an inhibition of the hunger center or an influence on the satiety center in the hypothalamus of the brain. The best known representatives of this group of substances are aminorex, cathine, ephedrine, phentermine, phenylpropanolamine (PPA) or norephedrine, fenfluramine, sibutramine, nicotine and rimonabant. Many of these drugs are no longer used therapeutically because prolonged use leads to dependence and can cause severe side effects.

It is known that by increasing the release of dopamine and serotonin in certain brain areas with simultaneous exposure to nutrients through the ingested orally consumable products (especially foods) and by inducing leptin receptor and serotonin receptor proteins, appetite can be influenced negatively and satiety positively, for example by nonivamide. In addition to influencing appetite and satiety, this can also have a positive effect on the consumer's mood at the same time. Thus, foods are already known that comprise dopamine and serotonin and have a mood-lifting effect.

In studies, it has been shown that pure capsaicin, the main pungent ingredient from the chili pepper (Capsicum anuum), can exhibit an appetite-reducing and satiety-enhancing effect (Smeets, A. J. P. G., Westerterp-Plantenga, M., Capsaicin, in weight control and slimming ingredients in Food Technologies, Cho, Susan S. (Ed.), 201-211, Wiley-Blackwell: Ames, Iowa, 2010). This observed effect is presumably due to the fact, that capsaicin provides a sensation of heat when ingested orally.

However, the use of capsaicin in food is not allowed in the European Union (was removed from the Community Flavoring List in 2004) because the genotoxic potential of the compound was evaluated negatively (European Food Safety Authority (EFSA), P., Italy, Opinion of the Scientific Committee on Food on Capsaicin. European Comission 2002, (SDF/CS/FLAV/FLAVOUR/8 ADD1 Final)). In addition, its use in food is often difficult because capsaicin has a low taste threshold (sensory detection threshold) and a high potency as a pungent (16,000,000 Scoville units, see http://en.wikipedia.org/wiki/Capsaicin; version of entry with latest changes as of Nov. 11, 2011, 9:02 pm). Capsaicin is also used almost exclusively in the form of a capsicum extract, partly due to the high price of the pure substance, which contains residues of other aromatic substances that taste or smell like capsicum, in addition to other pungents, and is therefore of limited suitability for widespread use.

In EP 2 614 727 A1, nonivamide (N-nonanoylvanillylamine) is described as an extremely effective flavoring agent that exhibits the desired effects described above, namely appetite-reducing and satiety-enhancing. However, the intrinsic pungency of the substance makes it problematic to use, especially in mild aromatic end-uses.

WO 2017/220168 A1 discloses alternative non-pungent tasting cinnamyl alcohol derivatives for use in a therapeutic and non-therapeutic method as an agent to reduce appetite, provide a feeling of satiety, reduce energy intake, reduce body weight, or as a mood elevator. However, these derivatives are not as effective as nonivamide and therefore must be taken at relatively high doses.

In EP 2 918 270 A1, alkene acid derivatives with a less pungent taste than nonivamide are proposed for use in a therapeutic and non-therapeutic procedure as agents to reduce appetite, provide a feeling of satiety, reduce energy intake or as mood elevators. However, the substances are currently hardly commercially available and cannot be isolated in sufficient quantities even from natural extracts. The efficacy of the aforementioned substances is also lower compared to nonivamide, which means a higher dosage with corresponding sensory effects.

The primary task of the present invention is therefore to provide a combination agent, in particular with respect to the known effects of nonivamide, which can be used in its action as an agent for reducing appetite and/or as an agent for imparting a feeling of satiety and/or as an agent for reducing energy intake and/or as an agent for reducing body weight, and which in particular produces significantly stronger effects at the same or lower dosage in comparison with, for example, nonivamide.

Furthermore, it was the task of the present invention to provide such a combination agent, in particular an orally consumable product, which already exhibits the above-mentioned effects in small amounts and which does not cause any unpleasant sensory notes or a secondary taste when consumed or can be used in such a way that it solves the task according to the invention while not or not significantly influencing the organoleptic properties of an orally consumable product (in particular a foodstuff, feedstuff or medicament) in which it can be used.

### SUMMARY OF THE INVENTION

The present problems are solved by the objects of the independent patent claims.

A first object of the present invention relates to a combination agent comprising or consisting of the following ingredients:
(a) one or more first saturating component(s), in particular nonivamide;
(b) added free arginine or its isomers or derivatives; and optional
(c) one or more second saturation component(s), in particular a protein or a protein hydrolysate; and/or
(d) one or more sensory active additive(s).

A second embodiment according to the invention relates to the medical or non-medical use of the combination agent.

Furthermore, the present invention relates to the use of the aforementioned combination agent for use as a means for
(i) Reduction of appetite; and/or
(ii) Providing a feeling of satiety; and/or
(iii) Reduction of energy intake; and/or
(iv) Reduction of body weight.

Finally, the present invention relates to the use of the combination agent for the preparation of an orally consumable preparation, in particular a foodstuff, a food supplement, a feedstuff and a medicament, or a food ingredient, as well as an orally consumable preparation or food ingredient comprising the combination agent according to the invention.

Further aspects of the present invention and preferred embodiments will be apparent from the following detailed description and, in particular, from the wording of the appended patent claims.

### FIGURES

FIG. 1 is a diagram showing the subjective feeling of hunger before and after administration of combination agents according to the invention.

FIG. 2 is a diagram showing ad libitum energy intake 140 min after administration of combination agents according to the invention.

FIG. 3 is a graph showing the hunger hormone ghrelin concentrations 15, 30, 60, 90, and 120 min after administration of combination agents according to the invention.

FIG. 4 is a correlation analysis between serotonin satiety parameters and gastric emptying.

### DETAILED DESCRIPTION OF THE INVENTION

In a first aspect, the present invention relates to a combination agent comprising or consisting of the following ingredients:
(a) one or more first saturating component(s), in particular nonivamide;
(b) added free arginine or its isomers or derivatives; and optional
(c) one or more second saturation component(s), in particular a protein or a protein hydrolysate; and/or
(d) one or more sensory active additive(s).

The present invention is based on the surprising finding that the combination agent according to the invention, in particular an orally consumable product, can be used to reduce appetite and provide a faster feeling of satiety, thereby simultaneously reducing energy intake and/or increasing the basal metabolic rate. The reduced energy intake and/or increase in basal metabolic rate ultimately lead to weight reduction.

In the following, the components (a) to (d) of the combination agent according to the invention according to the first aspect of the present invention as well as their preferred embodiments are described and can be combined with each other as desired, as far as technically useful.

Component (a)—One or More First Saturation Component(s)

The one or more first satiety component(s) of the combination agent according to the invention are substances with a sensory taste or odor value (aroma substances) with a simultaneous weight-reducing effect, the weight-reducing effect being based mainly on an inhibition of the hunger center or an influence on the satiety center in the hypothalamus of the brain. Furthermore, these substances can contribute to weight reduction with a sympathomimetic effect by increasing the basal metabolic rate. However, the effect may also preferentially be due to increases in peripheral plasma serotonin levels. Conversely, lower plasma serotonin levels have been associated with increased appetite (Hochkogler, Christina M. et al., Appetite-inducing effects of homoeriodictyol: Two randomized, cross-over interventions, Molecular nutrition & food research, 61.12 (2017): 1700459).

Such first satiating components used in the combination agent according to the invention are in the present context preferably naturally occurring low molecular weight substances such as nonivamide, capsaicin, trans-pellitorin, cinnamaldehyde, cinnamyl alcohol derivatives, nitrogen-containing cinnamic acid amide derivatives or aromatic alkenoic acid derivatives which increase the peripheral serotonin level. These satiety components thereby also influence lipid and/or glucose metabolism, gastric residence time of the bolus, and ultimately reduce the feeling of hunger and/or provide a long-lasting feeling of satiety.

The first satiety components have in common that they have a sensory active effect, in the combination agent according to the invention, however, the one or more first satiety components are used in an amount below or in the vicinity of their sensory recognition threshold (lowest concentration at which a taste or odor impression can be perceived) and thus not in aroma-imparting concentrations, so that no unpleasant sensory notes or an undesirable off-taste occur when the combination agent according to the invention is consumed. At the same time, however, the physiological effects described above and desired, namely appetite-reducing and satiety-increasing, are achieved in the concentration range used.

In all embodiments of the first saturation component, the first saturation component or a mixture of more than one of the first saturation components is preferred to exhibit only a subthreshold odor or taste value in the feed concentration, i.e., preferably the first saturation component or a mixture of more than one of the first saturation components is used around the detection threshold or lower.

In a preferred embodiment of the present invention, the first saturating component is nonivamide.

### Nonivamide:

Nonivamide or N-nonanoylvanillylamine (as described in EP 2 614 727 A1, which becomes part of this application by way of reference with respect to the corresponding compounds disclosed therein) is a known and approved flavoring agent (Flavis number 16.006; EFSA: has been evaluated as ‘no safety concern’ in FGE.86; CAS number 2444-46-4) and is already used in a variety of foods to achieve a distinct pungency. The irritation threshold of N-nonanoylvanillylamine is 0.054 mg/kg, the detection threshold 0.107 mg/kg (cf. Kollmannsberger, H., Ingredient composition and sensory quality of 20 cultivars of various Capsicum species, dissertation, Technical University of Munich, Weihenstephan, 2007, page 9).

N-nonanoylvanillylamine is also known as pseudocapsaicin, N-((4-hydroxy-3-methoxyphenyl-) methyl-) nonanamide, pelargonic acid vanillylamide or nonivamide. N-nonanoylvanillylamine (compound I) is shown in the following figure for clarity:

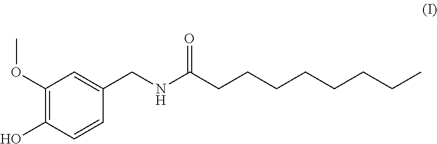 [SMILES: CCCCCCCCC(=O)NCc1ccc(O)c(OC)c1]

As already noted above with the example of capsaicin, the use of pungent-tasting compounds is not always possible without problems. Often, a pungency of orally consumable products (especially food, feed, and pharmaceuticals) is not desired, and orally consumable products that taste too pungent are often rejected by the consumer.

Surprisingly, N-nonanoylvanillylamine has been shown to be capable of already
in a concentration range from 0.01 μM to 10 μM (0.003 mg/kg to 3 mg/kg) stimulate serotonin release in neurons by up to about 270% against the respective control
   and
at a concentration of about 0.03 mg/kg (equivalent to 0.1 μM) stimulate dopamine release after acetylcholine stimulation by up to about 700% against the respective control,
   i.e. in each case in a concentration that is both well below the threshold values and far below typical use concentrations in orally consumable products (in particular foodstuffs, feedstuffs or pharmaceuticals).

The lipophilicity of N-nonanoylvanillylamine generally implies good bioavailability, i.e. good absorption from the gastrointestinal tract into the bloodstream. Thus, in particular, even at use concentrations of N-nonanoylvanillylamine below the taste recognition threshold, i.e. below 0.1 mg/kg, and at intake of usual consumption levels of orally consumable products (in particular food, feed and drugs), the above-mentioned biological effects occur in vivo.

Preferably, N-nonanoylvanillylamine is used in the combination agent at a concentration of 1 mg/kg or less, based on the total weight of the combination agent, preferably at a concentration of 0.5 mg/kg or less, more preferably at a concentration of 0.1 mg/kg or less, most preferably at a concentration of 0.05 mg/kg or less.

In another variation of the combination agent according to the first aspect of the present invention, the one or more first saturating components are selected from the group consisting of: Capsaicin, trans-pellitorin, cinnamaldehyde, cinnamyl alcohol derivatives, nitrogen-containing cinnamic acid amide derivatives, and aromatic alkene acid derivatives, and mixtures thereof. Preferably, these can also be used in combination with nonivamide (N-nonanoylvanillylamine), resulting in beneficial synergies in effects.

### Capsaicin:

Capsaicin is an alkaloid naturally occurring in various bell pepper species that causes a heat or pungent stimulus in mammals by acting on specific receptors and, associated with this, the release of neuropeptides such as substance P. Chemically, capsaicin is a fatty acid amide, more specifically the vanillylamide of the fatty acid trans-8-methyl-6-nonenoic acid. Capsaicin (compound II) is shown in the following figure for clarification:

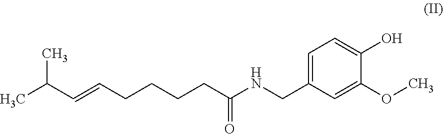 [SMILES: COc1cc(CNC(=O)CCCC/C=C/C(C)C)ccc1O]

Preferably, capsaicin is used in the combination agent at a concentration of 0.5 mg/kg or less, based on the total weight of the combination agent, preferably at a concentration of 0.25 mg/kg or less, more preferably at a concentration of 0.05 mg/kg or less, most preferably at a concentration of 0.025 mg/kg or less.

### Pellitorin:

Pellitorin (trans-pellitorin) is a main active ingredient of bertam root. It exhibits a strong anesthetic sensory effect known as “tingling” and strongly stimulates salivation. In small amounts, pellitorin is used as a flavoring agent to mask astringency or increase juiciness. Pellitorin (compound III) is shown in the following figure for clarity:

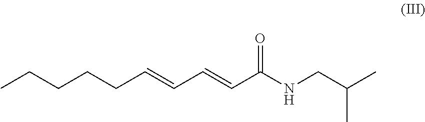 [SMILES: CCCCC/C=C/C=C/C(=O)NCC(C)C]

The detection threshold of pellitorin is 0.1 mg/kg.

Pellitorin is used in the combination agent according to the present invention in a concentration of 2 mg/kg or less, based on the total weight of the combination agent, preferably in a concentration of 0.2 mg/kg or less, more preferably in a concentration of 0.1 mg/kg or less. Preferably, pellitorin is used in a concentration of at least 0.0001 mg/kg or more, based on the total weight of combination agent, preferably in a concentration of 0.001 mg/kg or more, and most preferably in a concentration of 0.005 mg/kg or more.

### Cinnamaldehyde:

Cinnamaldehyde (3-phenyl-2-propenal) is a yellowish, intensely cinnamon-smelling, oily liquid. The sensory threshold value is below 1 ppm. It is the main flavoring agent of cinnamon bark. Cinnamaldehyde (compound IV) is shown in the following figure for clarification:

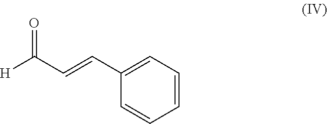

The effect on satiety or reduction of energy intake by cinnamaldehyde is well known and supported by studies in mice (e.g., Camacho S., Michlig S., de Senarclens-Bezençon C., Meylan J., Meystre J., Pezzoli M. et al., Anti-obesity and anti-hyperglycemic effects of cinnamaldehyde via altered ghrelin secretion and functional impact on food intake and gastric emptying, Sci. Rep. 2015; 5 (1): 7919).

According to another preferred embodiment of the present invention, cinnamaldehyde of formula (IV) is present as (E)- and/or (Z)-isomer, preferably in a mixture (in each case) more than 50%, preferably more than 90%, particularly preferably more than 95% in the E-configuration.

Preferably, cinnamaldehyde of formula (IV) is used in the combination agent at a concentration of 100 mg/kg or less, based on the total weight of the combination agent, preferably at a concentration of 50 mg/kg or less, more preferably at a concentration of 10 mg/kg or less, most preferably at a concentration of 1 mg/kg or less.

### Cinnamyl Alcohol Derivatives:

Another first saturating component used alternatively in the combination agent according to the present invention is a cinnamyl alcohol derivative of formula (V) (as described in WO 2017/220168 A1, which, with respect to the corresponding compounds disclosed therein, becomes part of this application by way of reference) or is a mixture of two or more cinnamyl alcohol derivatives of formula (V):

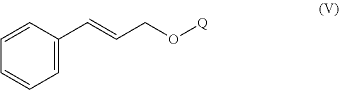

wherein the double bond may have an E or Z configuration or any mixture of E and Z configurations, and wherein Q is hydrogen or an acyl group of formula (VI)

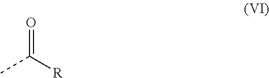

wherein R is hydrogen or methyl or a saturated or unsaturated, linear or branched or cyclic aliphatic moiety having 2 to 6 carbon atoms or phenyl or phenylmethyl.

According to a preferred embodiment, in the cinnamyl alcohol derivative of formula (V) or the mixture of two or more cinnamyl alcohol derivatives of formula (V), R is (in the case of the presence of a mixture, each independently) selected from the group consisting of hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl, 3-methyl-3-pentyl, vinyl, E- and Z-1-propen-1-yl, 2-propen-1-yl, E- and Z-2-buten-2-yl, 3-buten-2-yl, E- and Z-prenyl, E- and Z-isoprenyl, phenyl and phenylmethyl.

Particularly preferred according to the invention is the cinnamyl alcohol derivative of formula (V) selected from the group consisting of:

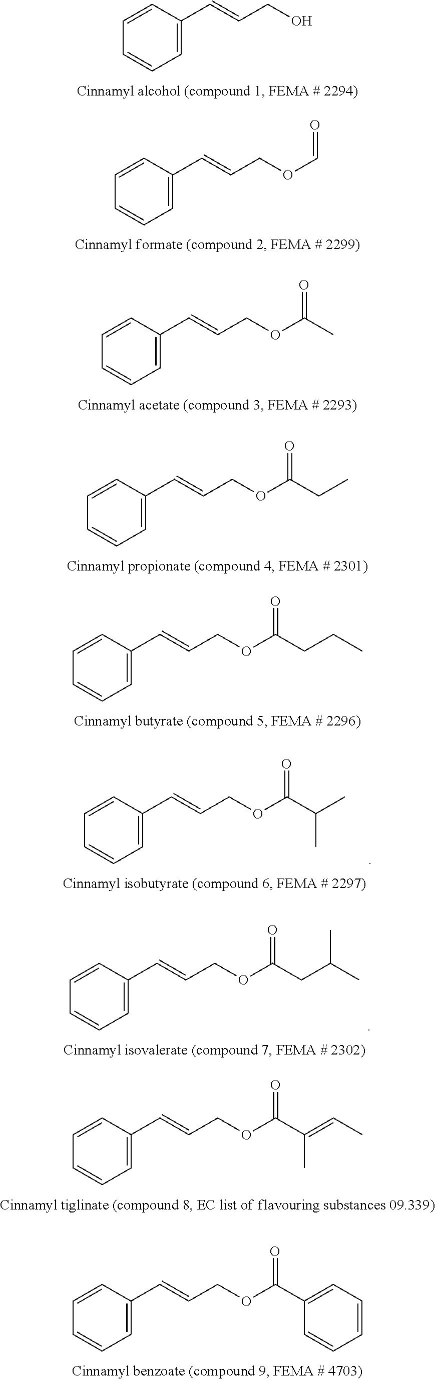

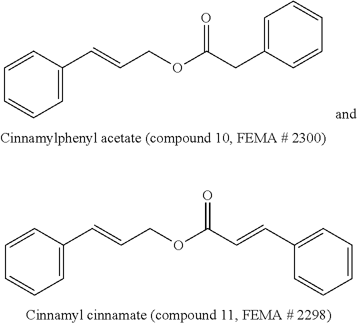 [SMILES: O=C(Cc1ccccc1)OC/C=C/c1ccccc1 ; O=C(/C=C/c1ccccc1)OC/C=C/c1ccccc1]

According to a further preferred embodiment of the present invention, the cinnamyl alcohol derivative of formula (V) or the cinnamyl alcohol derivatives of formula (V) are present in the mixture (in each case) in the E configuration to an extent of more than 50%, preferably more than 90%, particularly preferably more than 95%.

The combination agent according to the first aspect of the present invention preferably comprises a cinnamyl alcohol derivative of formula (V) or a mixture of at least two, three, four, five, six, seven, eight, nine, ten or eleven cinnamyl alcohol derivatives of formula (V), which are preferably selected from the group of compounds consisting of: compound 1, compound 2, compound 3, compound 4, compound 5, compound 6, compound 7, compound 8, compound 9, compound 10 or compound 11. It is also advantageous if all 11 compounds are present in such a combination agent. Compounds 1 and 2 are preferred in this respect.

The cinnamyl alcohol derivatives of formula (V) to be used according to the invention are capable of stimulating serotonin release in neurons up to about 130% of the positive control (nonivamide, EP 2 614 727 A1) already in a concentration range of 10 μM (about 1 mg/kg to about 3 mg/kg). The respective concentrations of the aromatic cinnamyl alcohol derivatives are thus low compared to typical use concentrations to achieve a flavor effect. Although flavor effects are to be expected, these should not be determinant even in mildly flavored orally consumable products.

According to a preferred embodiment, the total concentration of the cinnamyl alcohol derivatives of formula (V) in the combination agent is 20 mg/kg or less, preferably 5 mg/kg or less, particularly preferably 2 mg/kg or less, in each case based on the total weight of the combination agent. When determining the total concentration of the cinnamyl alcohol derivatives in the mixture, the sum of the concentrations of the individual compounds is meant.

### Nitrogen-Containing Cinnamic Acid Amide Derivatives:

In another variation of the present invention, the one first saturating component is a nitrogen-containing cinnamic acid amide derivative of formula (VII) (as described in EP 2 737 807 A1, which, with respect to the corresponding compounds disclosed therein, becomes part of this application by way of reference) or a mixture of two or more nitrogen-containing cinnamic acid amide derivatives of formula (VII):

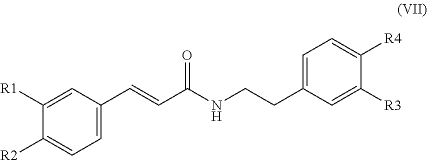

wherein R1, R2, R3 and R4 independently represent hydrogen or a methoxy group.

Among the nitrogen-containing cinnamic acid amide derivatives to be used according to the invention, those in which at least one of the groups R1, R2, R3 and R4 in formula (VII) represents hydrogen have proved to be particularly advantageous. Particularly preferred is the use of cinnamic acid amide derivatives in which R1, R2 and R4 in formula (VII) stand for a methoxy group and R3 stands for hydrogen.

The combining agent according to the first aspect of the present invention preferably comprises a nitrogen-containing cinnamic acid amide derivative of formula (VII) or a mixture of at least two, three, four, five or more nitrogen-containing cinnamic acid amide derivatives of formula (VII).

According to a preferred embodiment, the total concentration of the cinnamic acid amide derivatives of the formula (VI) in the combination agent is 100 mg/kg or less, preferably 50 mg/kg or less, particularly preferably 5 mg/kg or less, in each case based on the total weight of the combination agent. When determining the total concentration of the nitrogen-containing cinnamic acid amide derivatives in mixture, the sum of the concentrations of the individual compounds is meant.

### Aromatic Alkene Acid Derivatives:

Further, the one first saturating component is an aromatic alkenoic acid derivative of formula (VIII) (as described in EP 2 918 270 A1 and EP 2 918 271 A1, which, with respect to the corresponding compounds disclosed therein, become part of this application by way of reference) or mixtures of two or more nitrogen-containing cinnamic acid amide derivatives of formula (VIII):

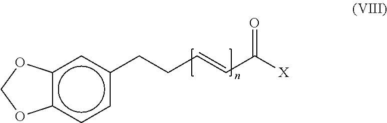

wherein
n=1, 2, wherein the resulting double bond(s) being independently present as E- or Z-configuration,
X=—OR¹ or —NR²R³,
R¹ is selected from the group consisting of: Hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl or isoprenyl, and
R², R³ are independently selected from the group consisting of: Hydrogen, methyl, ethyl, propyl, 2-propyl, 1-butyl, 2-butyl, 2-methyl-1-propyl, 1-pentyl, 2-pentyl, 3-pentyl, 2-methyl-1-butyl, 2-methyl-2-butyl, 2-methyl-3-butyl, 3-methyl-1-butyl, 1-hexyl, 2-hexyl, 3-hexyl, 2-methyl-1-pentyl, 2-methyl-2-pentyl, 2-methyl-3-pentyl, 2-methyl-4-pentyl, 3-methyl-1-pentyl, 3-methyl-2-pentyl or 3-methyl-3-pentyl, allyl, prenyl, isoprenyl, or
R² and R³ together form a saturated or unsaturated carbocyclic ring selected from the group consisting of: —CH2-CH2-, —CH2-CH2-CH2-, —CH2-CH2-CH2-CH2-, —CH═CH—CH═CH—, —CH2-CH2-CH2-CH2-CH2-, —CH═CH—CH2-CH2-CH2-, —CH2-CH═CH—CH2-CH2-, —CH═CH—CH═CH—CH2-, —CH═CH—CH═CH—CH═.

Particularly preferred here are the aromatic alkene acid derivatives of formula (VIII) selected from the group consisting of:

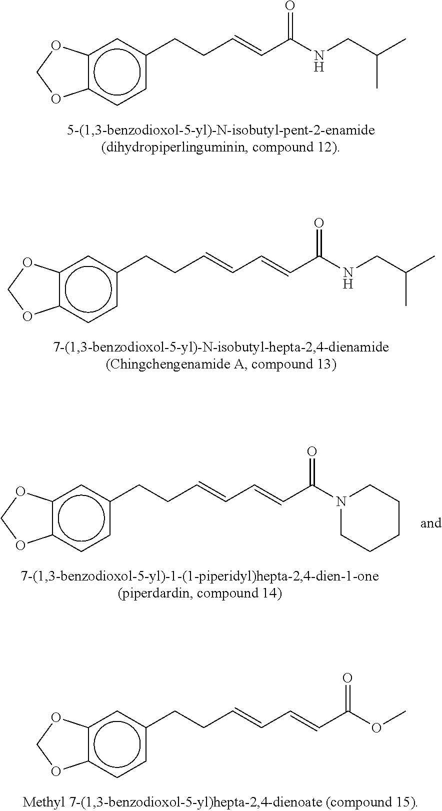

The aromatic alkenoic acid derivatives of formula (VIII) according to the invention are preferably more than 50%, particularly preferably more than 80%, especially preferably more than 90%, in the E configuration, the aromatic alkenoic acid derivatives preferably being selected from compounds 12 to 15.

The combination agent according to the first aspect of the present invention preferably comprises an aromatic alkenoic acid derivative of formula (VIII) or a mixture of at least two, three or more aromatic alkenoic acid derivatives of formula (VIII), which are preferably selected from the group consisting of compound 12, compound 13, compound 14 and compound 15. It is also advantageous if all four compounds are present in such a combination agent.

The aromatic alkene acid derivatives of formula (VIII) according to the invention are capable of stimulating serotonin release in neurons by a maximum of up to about 300% against the respective control already in a concentration range of 0.01 μM to 10 μM (about 0.02 mg/kg to about 2 mg/kg).

The respective concentrations of the aromatic alkene acid derivatives are low compared to typical feed concentrations to achieve a flavor effect; thus, strongly perceptible flavor effects are not expected.

Accordingly, a preferred embodiment is a combination agent in which the total concentration of the aromatic alkene acid derivatives of formula (VIII) is present in a range of 20 mg/kg or less, preferably in a concentration of 5 mg/kg or less, particularly preferably in a concentration of 2 mg/kg or less, based on the total weight of the combination agent. When determining the total concentration of aromatic alkene acid derivatives in the mixture, the sum of the concentrations of the individual compounds is meant.

A characteristic feature of the first satiating component(s) described above and used in the combination agent according to the present invention in accordance with the invention is that they reduce the feeling of hunger or act against cravings, increase the feeling of satiety and/or stimulate the metabolism.

Most preferably, nonivamide (N-nonanoylvanillylamine) alone or in combination with capsaicin, cinnamaldehyde and/or trans-pellitorin is used as the first satiety component in the combination agent according to the present invention, for example nonivamide and capsaicin and/or cinnamaldehyde and/or trans-pellitorin, or capsaicin and cinnamaldeyhd and/or trans-pellitorin, or cinnamaldehyde and trans-pellitorin. Due to the serotonin-releasing effect, the feeling of hunger can be particularly strongly reduced with such combinations as the first satiety components.

If several first saturation components are used in a mixture, the respective first saturation components to be used according to the invention can each be used at the above-mentioned total concentration in the combination agent according to the invention in individual concentrations far, i.e. well, below their respective sensory recognition threshold value with a constant effect according to the invention without negatively influencing the taste. Since some of the above-mentioned first saturation components have a sensory recognition threshold value lower than that of nonivamide while retaining the same effect according to the invention, they can be used in the combination agent according to the invention in a higher amount or can replace part of the nonivamide in order thus to reduce its concentration below its sensory recognition threshold value.

Accordingly, a preferred embodiment is a combination agent in which nonivamide is used in combination with capsaicin and/or cinnamaldehyde and/or trans-pellitorin in individual concentrations which, in the use concentration, i.e. the total concentration in the combination agent according to the invention, exhibit only a subthreshold aroma value, without deterioration of the physiological effects described above.

The (total) concentration of the first saturation component is understood to be either the concentration of a first saturation component in the combination agent according to the invention when this is used alone, or the sum of the individual concentrations of the respective individual first saturation components when these are used in mixture in the combination agent according to the invention.

Some of the first saturating components previously described in detail, for example capsaicin, occur naturally in certain plant species, and some of the compounds are obtained and used either directly, i.e., as pure compounds, or in the form of extracts and preparations. For example, compound 12 was found, e.g., in Piper longum (stick pepper, Tabuneng W., Bando H., Amiya T., 1983, Chemical & Pharmaceutical Bulletin, 31, 3562-3565), compound 13 in P. nigrum (Friedman M., Levin C. E., Lee S. U., Lee J. S., Ohnisi-Kameyama M., Kozukue N., 2008, J. Agric. Food Chem., 56, 3028-3036) and compound 14 in Piper nigrum (black pepper, Kapoor I. P., Singh B., Singh G., De Heluani C. S., De Lampasona M. P., Catalan C. A., 2009, J. Agric. Food Chem., 57, 5358-5364) described.

Such extracts can be obtained from the corresponding fresh or dried plants or parts of plants, leaves, seeds or fruit bodies. Usually, the dried plant parts (e.g., fresh or dried roots, fruits, seeds, bark, wood, stems, leaves or flower [parts]) are extracted for this purpose, preferably in comminuted form, with a solvent suitable for food and beverages at temperatures from 0° C. to the boiling point of the respective solvent or solvent mixture, then filtered and the filtrate wholly or partially concentrated, preferably by distillation, freeze-drying or spray-drying. The crude extract thus obtained can then be further processed, for example treated enzymatically, treated with acid (e.g. under pressure), with acidic ion exchangers or with steam, usually at pressures of 0.01 mbar to 100 bar, preferably at 1 mbar to 20 bar, and/or taken up in a solvent suitable for food and beverages.

Preferred extracts are those in which the above-described first saturation components represent the major portion based on the total amount of extracted compounds, for example 1 to 90% by weight, preferably 10 to 80% by weight, even more preferably 20 to 70% by weight based on the total amount of extracted compounds.

In another embodiment, the combination agent according to the first aspect of the present invention thus comprises a plant extract, e.g., from Capsicum species, comprising at least one of the saturating component(s) described above.

Ingredient (b)—Added Free Arginine or its Isomers or Derivatives.

As another main ingredient, the combination agent according to the first aspect of the present invention comprises added free arginine or isomers or derivatives thereof.

Surprisingly, it was found that in addition to the one or more first satiety component(s), the addition of free arginine in the combination agent according to the invention results in a further synergistically greater reduction in appetite and/or imparting a feeling of satiety compared to a preparation comprising only one or more of the first satiety component(s) described above or only arginine.

By added free arginine is meant a free, i.e. not bound, amino acid which is added to the combination agent and is not selectively released from other protein components of the combination agent or does not originate from other protein components of the combination agent, in particular not from the second saturation component of the combination agent as described further below.

The oral administration of certain free amino acids can lead to a stimulation of the release of the satiety hormones GLP-1 and PYY- and thus to a decrease in energy intake. However, the effect described here for the amino acid arginine significantly exceeds these stimulation effects.

The amino acid arginine can be present in the D and L configuration according to the possible stereoisomers. The combination agent according to the present invention thus also includes the possible stereoisomers of the above-mentioned amino acid as well as mixtures of the stereoisomers.

In a preferred embodiment, the amino acid arginine in the D or L configuration or mixtures thereof is used in the combination agent according to the invention, even more preferably in the enantiomerically pure L configuration.

The amino acid arginine can also be used in the form of its derivatives in the combination agent according to the invention, in particular in the form of its salts or mixtures thereof. Preferred are the monovalent or divalent salts and the ammonium salt. Among the salts, sodium, calcium-, magnesium, potassium or ammonium salts are particularly preferred.

Due to their influence on proton secretion, as evidenced by the HGT-1 parietal cell model, surprisingly, of the amino acids listed in Table 1 below, L-arginine and D-arginine are particularly capable of reducing hunger and inducing a sustained feeling of satiety. This effect is illustrated by the intracellular proton index (IPX) values listed below in Table 1.

TABLE 1

| | | | molecular | IPX | | P-value |
|---|---|---|---|---|---|---|
| amino | | functional | weight | [induced by 50 mM AA] | | (stereo- |
| acid | chemical structure | group | [g/mol] | L | D | isomerism) |
| TRP | 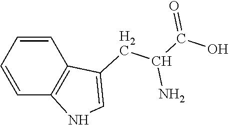 [SMILES: NC(Cc1c[nH]c2ccccc12)C(=O)O] | aromatic | 204.2 | −0.66 ± 0.23 | −0.04 ± 0.04 | <0.001 |
| PHE* | 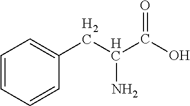 [SMILES: NC(Cc1ccccc1)C(=O)O] | aromatic | 165.2 | 0.50 ± 0.05 | 1.58 ± 0.10 | <0.001 |
| ARG* | 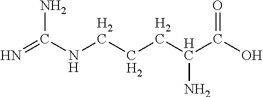 [SMILES: N=C(N)NCCCC(N)C(=O)O] | guanidino | 174.2 | −2.71 ± 0.11 | −2.37 ± 0.05 | <0.05 |
| ILE* | 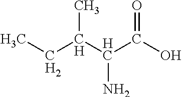 | aliphatic | 131.2 | 0.58 ± 0.07 | −0.40 ± 0.04 | <0.001 |
| VAL* | 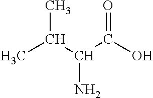 | aliphatic | 117.2 | 0.99 ± 0.05 | −0.44 ± 0.17 | <0.001 |
| LEU* | 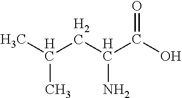 | aliphatic | 131.2 | 1.38 ± 0.14 | 1.10 ± 0.13 | <0.05 |
| PRO* | 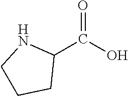 | imino acid | 115.1 | 0.44 ± 0.08 | 0.63 ± 0.09 | p = 0.156 |
| THR* | 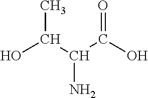 | hydroxyl | 119.1 | 1.43 ± 0.10 | 1.57 ± 0.05 | p = 0.351 |
| SER* | 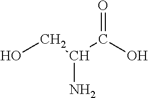 | hydroxyl | 105.1 | 1.40 ± 0.12 | 0.87 ± 0.10 | <0.01 |
| ALA* | 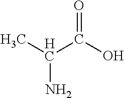 | aliphatic | 89.1 | 0.87 ± 0.10 | −0.35 ± 0.08 | <0.001 |

The value of intracellular proton secretion (IPX) in the parietal cell model HGT-1 (proton secretion assay), which is a marker for gastric secretion, indicates the extent to which the amino acid is able to inhibit proton secretion and is thus indirectly a measure of saturation. The intracellular proton index (IPX) is based on log 2 data of intracellular proton concentration. The lower the IPX, the more protons are secreted. A low IPX is interpreted as stimulation of gastric acid, which in turn is a saturation signal.

Of the amino acids listed in Table 1, L-arginine and D-arginine exhibit the best acid modulating effect, which is an indicator of satiety.

The best results for reducing hunger pangs and rapidly achieving satiety are obtained when the combination agent according to the first aspect of the present invention comprises added free L-arginine or D-arginine or mixtures thereof.

The best results of a hunger-reducing and satiety-enhancing effect are obtained when the combination agent according to the first aspect of the present invention comprises added free L-arginine.

Particularly advantageous, i.e. effective, is the combination agent comprising the added free amino acid L-arginine or D-arginine or mixtures of the aforementioned amino acids in an amount of from 0.1 to 20% by weight, preferably from 5 to 20% by weight, based on the total weight of the combination agent. Particularly preferred is an amount of added free amino acid L-arginine or D-arginine or mixtures of the aforementioned amino acids of from 0.5 to 10% by weight and most preferably from 2 to 7% by weight, based on the total weight of the combination agent.

The very best effects according to the invention are achieved with a combination agent comprising the added free amino acid L-arginine in an amount of from 0.1 to 20% by weight, preferably from 5 to 20% by weight, based on total weight of the combination agent. Particularly preferred is an amount of added free amino acid L-arginine of from 0.5% to 10% by weight, and most preferably from 2% to 7% by weight, based on total weight of the combination agent.

Optional Component (c)—One or More Second Saturation Component(s).

Particularly advantageous, i.e. effective, is the combination agent according to the first aspect of the present invention, in particular an orally consumable product optionally comprising as a further component one or more second saturating component(s).

Surprisingly, it was found that when the combination agent with a second satiety component was administered, appetite could be reduced even more and a feeling of satiety occurred even more quickly compared with administration of the same combination agent without a second satiety component.

Like the first satiety component of the combination agent according to the invention, the second satiety component is also a component suitable for consumption with a weight-reducing effect, the weight-reducing effect being based mainly on inhibition of the hunger center or on influencing the satiety center in the hypothalamus of the brain. Furthermore, this ingredient with a sympathomimetic effect may contribute to weight reduction by increasing the basal metabolic rate.

The second saturating component is in particular edible animal or vegetable proteins, in particular protein isolates, or edible animal or vegetable proteins in the form of fractions, partial or complete hydrolysates, or intermediates produced by physicochemical processes or fermentative or enzymatic treatment of the proteins.

In a preferred embodiment, the combination agent according to the invention comprises an edible animal or vegetable protein, in particular a protein isolate, as the one or more second saturating component(s). The edible animal or vegetable protein or protein isolate is selected from proteins of the group consisting of: Meat (mammals, birds, reptiles, amphibians, fish), crustaceans, shellfish, mussels, molluscs, insects, eggs, milk, in particular casein and whey, algae, cereals, in particular wheat, barley, rapeseed, sunflower, rice, potatoes, corn, soy, beans, peas, lentils, lupins, peanut, alfalfa, hemp and other proteins from edible plants.

The protein isolates may contain between 30 and 100% pure protein, preferably 50 to 95, more preferably 70 to 90% pure protein, and are obtained by conventional methods.

In a further preferred variant of the combination agent, the one or more second saturating components are protein hydrolysate(s) selected from the group consisting of partial hydrolysate or complete hydrolysate of proteins from meat (mammals, birds, reptiles, amphibians, fish), crustaceans, shellfish, mussels, molluscs, insects, eggs, milk, especially casein and whey, algae, cereals, especially wheat, barley, rapeseed, sunflower, rice, potatoes, corn, soybean, beans, peas, lentils, lupins, peanut, alfalfa, hemp and other proteins from edible plants.

Protein hydrolysates are proteins cleaved by chemical or enzymatic treatment. Depending on the size of the protein fragments, a distinction is made between partial hydrolysate, a mixture of peptides of different chain length and amino acids, and complete hydrolysate, a mixture of monomeric amino acids.

Starting products for the preparation of such protein isolates, fractions, partial or complete hydrolysates are edible animal or vegetable proteins selected from the group consisting of: Meat (mammals, birds, reptiles, amphibians, fish), crustaceans, shellfish, mussels, molluscs, insects, eggs, milk, especially casein and whey, algae, cereals, especially wheat, barley, rapeseed, sunflower, rice, potatoes, corn, soybean, beans, peas, lentils, lupins, peanut, alfalfa, hemp and other proteins from edible plants.

Particularly preferred are protein hydrolysates with a degree of hydrolysis of at least 10%, preferably at least 30%, especially preferably greater than 50% of the amino acids bound in the protein portion.

Protein hydrolysates produced by peptidases/hydrolases showing a preference for cleavage at N- and/or C-specific arginine positions in the protein and thereby achieving selective enrichment in L-arginine or L-arginine-containing small peptides, such as described in Schindler A., Dunkel A., Stähler F., Backes M., Ley J., Meyerhof W., Hofmann T., Discovery of salt taste enhancing arginyl dipeptides in protein digests and fermented fish sauces by means of a sensomics approach, J. Agric. Food Chem., 2011, 59 (23), 12578-12588, are seen as a particularly preferred embodiment.

Particularly beneficial effects of weight reduction were surprisingly found when a protein or a protein isolate with an intrinsically high L-arginine content or a protein hydrolysate from a protein with an intrinsically high L-arginine content is used as a second satiating component in the combination agent according to the present invention.

Proteins with intrinsically high L-arginine content include proteins from pumpkin seeds (5353 mg L-arginine per 100 g), walnuts, peanuts (3460 mg L-arginine per 100 g), almonds (2750 mg L-arginine per 100 g), Pine nuts (2413 mg L-arginine per 100 g), lentils (2240 mg L-arginine per 100 g), peas, chickpeas, hazelnuts (2030 mg L-arginine per 100 g), shrimp (1740 mg L-arginine per 100 g), Beef (1540 mg L-arginine per 100 g), Pork, Chicken Breast (1350 mg L-arginine per 100 g), Salmon (1330 mg L-arginine per 100 g), Edam (1030 mg L-arginine per 100 g), Buckwheat (970 mg L-arginine per 100 g), Corn, Rice, Eggs (840 mg L-arginine per 100 g), and Cow's Milk (all above content data are based on 100 g of the respective food).

In a particularly preferred embodiment according to the present invention, the combination agent therefore comprises, as a second saturating component, a protein or protein isolate with a high intrinsic L-arginine content or a protein hydrolysate from a protein with a high intrinsic L-arginine content.

Most preferred is a protein or protein isolate or protein hydrolysate as a second saturating component comprising an intrinsic L-arginine content of at least 800 mg L-arginine per 100 g, even more preferred of at least 1000 mg L-arginine per 100 g and most preferred of at least 2500 mg L-arginine per 100 g food.

The amount of the one or more second saturating components in the combination agent comprising components (a), (b) and (c) is from 1 to 95% by weight, preferably from 1 to 80% by weight, and more preferably from 5 to 75% by weight, based on the total weight of the combination agent.

A low intrinsic L-arginine content of the protein or protein isolate or protein hydrolysate used as the second saturation component can be compensated for or advantageously increased by the addition of free L-arginine. This is done, on the one hand, by the addition of free L-arginine via component (b) of the combination agent or by enrichment of the protein or protein isolate or protein hydrolysate with free L-arginine via component (c).

In the latter advantageous variant of the combination agent according to the first aspect of the present invention, the total content of L-arginine in the combination agent is therefore higher than the natural intrinsic L-arginine content of the protein component (protein or protein isolate or protein hydrolysate) of the second saturation component. The total content of L-arginine in the combination agent is thus a sum of the content of added free L-arginine (component (b)) plus the natural intrinsic L-arginine content of the protein component of the second saturation component (component (c)).

The amount of added free L-arginine in the combination agent comprising ingredients (a), (b) and (c) is preferably 0.1 to 20 wt %, more preferably 5 to 20 wt %, based on the total weight of the combination agent. Particularly preferred is an amount of added free L-arginine of 0.5 to 10% by weight, and most preferably 2 to 7% by weight, based on the total weight of the combination agent, in order to achieve a hunger-reducing and satiety-enhancing effect.

In order for the effect according to the invention to occur, advantageously the total content of L-arginine in the combination agent according to the invention in a regularly consumable amount (serving size) comprising components (a) and (b) or components (a), (b) and (c), is at least 0.1 g L-arginine, more preferably at least 0.5 g L-arginine and most preferably at least 1 g L-arginine, based on the total weight of the regularly consumable amount (serving size) of the combination agent.

Optional Ingredient (d)—One or More Sensory Active Substance(s)

In a further variant according to the first aspect of the present invention, the combination agent according to the invention optionally comprises one or more sensory active substance(s) selected from the group consisting of sweetening foods, sweeteners, flavoring agents and flavor modulators. With the addition of one or more of the aforementioned substance(s), the flavor or aroma profile of the combination agent according to the invention can be modified or balanced.

With the use of one or more sweetening food(s) as sensory active substance, a sweet impression can be created in the combination agent according to the invention. The sweetening foodstuff(s) used in the combination agent according to the invention are preferably selected from the group consisting of: liquid or dry concentrates, preferably powder, of sweet whey, sweet potato, honey, agave, acacia, fruit syrups, in particular of banana, grape, apple, apricot, date, sugar beet syrup and lactose.

With the addition of sweeteners, the combination agent according to the invention can be sweetened, for example, without increasing the energy content of the combination agent. In addition, the addition makes it possible, for example, to reduce or suppress an unpleasant taste, in particular a bitter taste, of the other components, for example of the first saturating component or of the free amino acid, of the combination agent.

The term “sweeteners” refers to such substances with a relative sweetening power of at least 25, based on the sweetening power of sucrose (which thus has a sweetening power of 1). Preferably, sweeteners to be used in the composition according to the invention are non-cariogenic and/or have an energy content of at most 5 kcal per gram of the composition according to the invention.

Advantageous sweeteners in a preferred composition according to the invention are selected from the following groups (a1) and (a2):
(a1) naturally occurring sweeteners, preferably selected from the group consisting of
(a1-1) Miraculin, monellin, mabinlin, thaumatin, curculin, brazzein, pentaidin, D-phenylalanine, D-tryptophan, glycine, D-threonine, D-serine and extracts or fractions obtained from natural sources containing these amino acids and/or proteins, and the physiologically acceptable salts of these amino acids and/or proteins, in particular the sodium, potassium, calcium or ammonium salts;
(a1-2) Neohesperidine dihydrochalcone, naringine dihydrochalcone, stevioside, steviol bioside, rebaudiosides, especially rebaudioside A, rebaudioside B, rebaudioside C, rebaudioside D, Rebaudioside E, Rebaudioside F, Rebaudioside G, Rebaudioside H, Rebaudioside M, Rebaudioside N, Rebaudioside O, Rebaudioside P, Rebaudioside R, Rebaudioside X, Rebaudioside Z, Dulcoside and Rubusoside, Suavioside A, Suavioside B, Suavioside G, Suavioside H, Suavioside I, Suavioside J, Baiyunoside 1, Baiyunoside 2, Phlomisoside 1, Phlomisoside 2, Phlomisoside 3 and Phlomisoside 4, Abrusoside A, Abrusoside B, Abrusoside C, Abrusoside D, Cyclocaryoside A and Cyclocaryoside I, Osladin, Polypodoside A, Strogin 1, Strogin 2, Strogin 4, Selligueain A, dihydroquercetin-3-acetate, perillartin, telosmoside A15, periandrin I-V, pterocaryosides, cyclocaryosides, mucurociosides, trans-anethol, Bryosides, Bryonosides, Bryonodulcosides, Carnosiflosides, Scandenosides, Gypenosides, Trilobatin, Phloridzin, Dihydroflavanols, Hematoxylin, Cyanine, Chlorogenic acid, albiziasaponin, telosmosides, gaudichaudioside, mogrosides, mogroside V, hernandulcines, monatin, phyllodulcin, glycyrrhetinic acid and their derivatives, in particular their glycosides such as glycyrrhizin, and the physiologically acceptable salts of these compounds, in particular the sodium, potassium, calcium or ammonium salts;
(a1-3) Extracts or enriched fractions of the extracts selected from the group consisting of Thaumatococcus extracts (Catemfestaude), extracts of Stevia ssp. (especially Stevia rebaudiana), Swingle extracts (Momordica or Siratia grosvenorii, Luo-Han-Guo), Glycerrhyzia ssp. extracts (especially Glycerrhyzia glabra), Rubus ssp. extracts (especially Rubus suavissimus), Citrus extracts and Lippia dulcis extracts; and
(a2) synthetic sweet tasting substances, preferably selected from the group consisting of magap, sodium cyclamate or other physiologically acceptable salts of cyclamic acid, acesulfame K or other physiologically acceptable salts of acesulfame, neohesperidine dihydrochalcone, naringine dihydrochalcone, saccharin, saccharin sodium salt, aspartame, superaspartame, neotame, alitame, advantame, perillartine, monelline, sucralose, lugduname, carrelam, sucrononate and sucrooctate.

In a further embodiment of the present invention, at least one flavoring substance is optionally used as a sensory active substance, in particular a flavoring, in the combination agent according to the invention in order to flavor the combination agent according to the invention. The aroma may be solid or liquid.

In a preferred embodiment, the combination agent according to the invention comprises a flavor to round off and/or refine its taste and/or smell of the composition. Suitable flavorings are preferably selected from the group consisting of synthetic, natural or nature-identical flavoring, smelling and flavoring agents and mixtures thereof, essential oils, reaction flavorings, smoke flavorings or other flavoring preparations. These may further preferably be protein [partial] hydrolysates, barbecue flavors, plant extracts, spices, spice preparations, vegetables and/or vegetable preparations, which may further contain suitable excipients and carriers. In particular, according to the invention, flavors or their components are preferably suitable which have a roasty, meaty (in particular chicken, fish, seafood, beef, pork, lamb, sheep, goat), vegetable (in particular tomato, onion, garlic, celery, leek, mushrooms, eggplant, seaweed), spicy (especially black and white pepper, chili, paprika, cardamom, nutmeg, allspice, mustard and mustard products), fried, yeasty, boiled, greasy, salty and/or spicy aroma impression and thus can enhance a spicy and salty impression.

Essential oils that can be a component of the aroma according to the invention are preferably selected from the group consisting of anise oil; valerian oil; basil oil; mugwort oil; bergamot oil; bitter almond oil; savory oil; bucco leaf oil; camphor oil; Cananga oil; Cardamom oil; Cascarilla oil; Cassia oil; Citronella oil; Citron oil; Coriander oil; Cumin oil; Dill herb oil; Dill seed oil; Tarragon oil; Eucalyptus citriodora oil; Eucalyptus oil; Fennel oil; Grapefruit oil; Ginger oil; Chamomile oil blue; Chamomile oil roman; Spearmint oil; Caraway oil; Lemongrass oil; Lovage oil; Lime oil distilled; Lime oil pressed; Bay leaf oil; Mace oil; Marjoram oil; Mandarin oil; Nutmeg oil; Clove leaf oil; Clove flower oil; Orange oil; Origanum oil; Parsley leaf oil; Parsley seed oil; Peppermint oil; Pepper oil; Allspice oil; Rosemary oil; Sage oil Dalmatian; Sage oil Spanish; Celery seed oil; Star anise oil; Thyme oil; Vanilla extract; Juniper berry oil; Wintergreen oil; Cinnamon leaf oil; Cinnamon bark oil and fractions thereof.

In the context of the present invention, reaction flavors that can be part of the flavor, which can be produced by roasting a spice or a pomace, are preferably also used, are preferably selected from the group consisting of flavors from roasting vegetable oils with potatoes, sweet potatoes, carrot pomace, onion pomace, sugar beet pomace, vegetable pomace, fruit pomace or mushroom pomace.

In the context of the present invention, preferred single flavoring agents to be used are selected from the group consisting of: Acetaldehyde, acethylmethylcarbinol, acetophenone, allyl capronate, alpha ionone, beta ionone, anisaldehyde, anisyl acetate, anisyl formate, benzaldehyde, benzothiazole, benzyl acetate, benzyl acetate, benzyl alcohol, benzyl benzoate, beta ionone, butyl butyrate, butyl capronate, butylidene phthalide, capric acid, caproic acid, caprylic acid, carvone, camphene caryophyllene, cineole, cinnamyl acetate, citral, citronellol, citronellal, citronellyl acetate, cyclohexyl acetate, cymol, Damascone, decalactone, diacetyl, dihydrocoumarin, dimethyl anthranilate, dimethyl anthranilate, dodecalactone, ethoxyethyl acetate, ethyl acetate, ethyl butyric acid, ethyl butyrate, ethyl caprinate, ethyl capronate, ethyl crotonate, ethyl formate, ethyl furaneol, ethyl guaiacol, ethyl isobutyrate, ethyl isovalerate, ethyl lactate, ethyl lactate, ethyl maltol, ethyl methyl butyrate, ethyl propionate, eucalyptol, eugenol, ethyl heptylate, 1-(4′-hydroxyphenyl)-2-butanone (Frambinor<®>), gamma-decalactone, geraniol, geranyl acetate, geranyl acetate, grapefruit aldehyde, methyl dihydrojasmonate (Hedion<®>), heliotropin, 2-heptanone, 3-heptanone, 4-heptanone, trans-2-heptenal, cis-4-heptenal, trans-2-hexenal, cis-3-hexenol, trans-2-hexenoic acid, trans-3-hexenoic acid, cis-2-hexenyl acetate, cis-3-hexenyl acetate, cis-3-hexenyl capronate, trans-2-hexenyl capronate, cis-3-hexenyl formate, cis-2-hexyl acetate, cis-3-hexyl acetate, trans-2-hexyl acetate, cis-3-hexyl formate, para-hydroxybenzylacetone, isoamyl alcohol, isoamyl isovalerate, Isobutyl butyrate, isobutyraldehyde, isoeugenol methyl ether, isopropyl methylthiazole, lauric acid, leavulinic acid, linalool, linalool oxide, linalyl acetate, maltol, I-menthol, menthofuran, methyl anthranilate, Methyl butanol, methyl butyric acid, 2-methyl butyl acetate, methyl capronate, methyl cinnamate, 5-methyl furfural, 3,2,2-methylcyclopentenolone, 6,5,2-methylheptenone, methyl dihydro jasmonate, methyl jasmonate, 2-methyl methyl butyrate, 2-methyl-2-pentenoic acid, methyl thiobutyrate, 3,1-methylthiohexanol, 3-methylthiohexyl acetate, nerol, nerylacetate, trans,trans,2,4-nonadienal, 2,4-nonadienol, 2,6-nonadienol, 2,4-nonadienol, nootkatone, delta octalactone, gamma octalactone, 2-octanol, 3-octanol, 1,3-octenol, 1-octyl acetate, 3-octyl acetate, palmitic acid, paraldehyde, phellandrene, pentanedione, phenylethyl acetate, phenyl ethyl alcohol, phenyl ethyl alcohol, phenyl ethyl isovalerate, piperonal, propionaldehyde, propyl butyrate, pulegone, pulegol, sinensal, sulfurol, terpinene, terpineol, terpinolene, 8,3-thiomenthanone, 4,4,2-thiomethylpentanone, thymol, delta-undecalactone, gamma-undecalactone, valencene, valeric acid, vanillin, acetoin, ethylvanillin, ethylvanillin isobutyrate (3-ethoxy-4-isobutyryloxybenzaldehyde), furaneol<®> (2,5-dimethyl-4-hydroxy-3 (2H)-furanone) and its derivatives (thereby preferably homofuraneol (2-ethyl-4-hydroxy-5-methyl-3 (2H)-furanone), homofuronol (2-ethyl-5-methyl-4-hydroxy-3 (2H)-furanone and 5-ethyl-2-methyl-4-hydroxy-3 (2H)-furanone), maltol and maltol derivatives (preferably ethylmaltol), coumarin and coumarin derivatives, gamma-lactones (preferably gamma-undecalactone, gamma-nonalactone, gamma-decalactone), delta-lactones (preferably 4-methyldeltadecalactone, massoilactone, deltadecalactone, tuberolactone), methyl sorbate, divanillin, 4-hydroxy-2 (or 5)-ethyl-5 (or 2)-methyl-3 (2H)-furanone, 2-hydroxy-3-methyl-2-cyclopentenone, 3-hydroxy-4,5-dimethyl-2 (5H)-furanone, acetic acid isoamyl ester, butyric acid ethyl ester, butyric acid n-butyl ester, Butyric acid isoamyl ester, 3-methylbutyric acid ethyl ester, n-hexanoic acid ethyl ester, n-hexanoic acid allyl ester, n-hexanoic acid n-butyl ester, n-octanoic acid ethyl ester, ethyl 3-methyl-3-phenyl glycidate, ethyl 2-trans-4-cis-decadienoate, 4-(p-hydroxyphenyl)-2-butanone, 1,1-dimethoxy-2,2,5-trimethyl-4-hexane, 2,6-dimethyl-5-heptene-1-al and phenylacetaldehyde, 2-methyl-3-(methylthio)-furan, 2-methyl-3-furanthiol, bis(2-methyl-3-furyl) disulfide, furfuryl mercaptan, methional, 2-acetyl-2-thiazoline, 3-mercapto-2-pentanone, 2,5-dimethyl-3-furanthiol, 2,4,5-trimethylthiazole, 2-acetylthiazole, 2,4-dimethyl-5-ethylthiazole, mercapto-3-methyl-1-butanol 2-acetyl-1-pyrroline, 2-methyl-3-ethylpyrazine, 2-ethyl-3,5-dimethylpyrazine, 2-ethyl-3,6-dimethylpyrazine, 2,3-diethyl-5-methylpyrazine, 3-isopropyl-2-methoxypyrazine, 3-isobutyl-2-methoxypyrazine, 2-acetylpyrazine, 2-pentylpyridine, (E,E)-2,4-decadienal, (E,E)-2,4-nonadienal, (E)-2-octenal, (E)-2-nonenal, 2-undecenal, 12-methyltridecanal, 1-penten-3-one, 2,3-butanedione, 4-hydroxy-2,5-dimethyl-3 (2H)-furanone, guaiacol, 3-hydroxy-4,5-dimethyl-2 (5H)-furanone, 3-hydroxy-4-methyl-5-ethyl-2 (5H)-furanone, dimethyl sulfide, trimethylamine, cinnamaldehyde, cinnamalcohol, methyl salicylate, isopulegol and—as far as possible-their cis/trans isomers, stereoisomers, enantiomers, diastereomers and epimers.

In addition, the flavors used according to the invention may be those preferably selected from the group consisting of egg, butter, toast, caramel and popcorn.

Preferably, the flavorings to be used according to the invention contain more than one of said flavoring agents, preferably two, three, four, five or more of said flavoring agents.

In a further embodiment of the present invention, at least one taste modulator is optionally used in the combination agent according to the invention. Taste modulators are those substances capable of masking or reducing an unpleasant (bitter, metallic, chalky, sour, astringent, pungent) taste sensation, enhancing or creating a pleasant taste sensation (sweet, salty, umami or tingling “tingling” or cooling effect) or correcting a taste sensation. Thus, a modulation of the taste of the composition according to the invention can be achieved.

These further modulating aroma and/or flavor substances (flavor modulators) as sensory active substance can be selected from the following lists:

Trans-tert-butylcyclohexanol (as described in WO 2009/087242, which becomes part of this application by way of reference with respect to the corresponding compounds disclosed therein); monosodium glutamate, glutamic acid; nucleotides, such as adenosine 5′-monophosphate, cytidine 5′-monophosphate, inosine 5′-monophosphate, guanosine 5′-monophosphate and pharmaceutically acceptable salts thereof; lactisols; Sodium salts, such as sodium chloride, sodium lactate, sodium citrate, sodium acetate, sodium gluconate; hydroxyflavanones, such as eriodictyol, sterubin (eriodictyol-7-methyl ether), homoeriodictyol and their sodium, potassium, calcium, magnesium or zinc salts (in particular those as described in EP 1 258 200, which, with respect to the corresponding compounds disclosed therein, becomes part of this application by way of reference); Hydroxybenzoic acid amides, such as 2,4-dihydroxybenzoic acid vanillylamide, 2,4-dihydroxybenzoic acid N-(4-hydroxy-3-methoxybenzyl)-amide, 2,4,6-trihydroxybenzoic acid-N-(4-hydroxy-3-methoxybenzyl)-amide, 2-hydroxybenzoic acid-N-4-(hydroxy-3-methoxybenzyl)-amide, 4-hydroxybenzoic acid-N-(4-hydroxy-3-methoxybenzyl)-amide, 2,4-dihydroxybenzoic acid-N-(4-hydroxy-3-methoxybenzyl)-amide monosodium salt, 2,4-dihydroxybenzoic acid-N-2-(4-hydroxy-3-methoxyphenyl)-ethylamide, 2,4-dihydroxybenzoic acid-N-(4-hydroxy-3-ethoxybenzyl)-amide, 2,4-dihydroxybenzoic acid-N-(3,4-dihydroxybenzyl)-amide, and 2-hydroxy-5-methoxy-N-[2-(4-hydroxy-3-methoxyphenyl)-ethyl]-amide); 4-hydroxybenzoic acid vanillylamide (in particular, those as described in WO 2006/024587, which, with respect to the corresponding compounds disclosed therein, becomes part of this application by way of reference); hydroxydeoxybenzoins, such as 2-(4-hydroxy-3-methoxyphenyl)-1-(2,4,6-trihydroxyphenyl)-ethanone, 1-(2,4-dihydroxyphenyl)-2-(4-hydroxy-3-methoxy-phenyl)-ethanone, 1-(2-hydroxy-4-methoxyphenyl)-2-(4-hydroxy-3-methoxyphenyl)-ethanone) (in particular those as described in WO 2006/106023, which, with respect to the corresponding compounds disclosed therein, becomes part of this application by way of reference); Hydroxyphenylalkanediones, such as gingerdione-[2], gingerdione-[3], gingerdione-[4], dehydrogingerdione-[2], dehydrogingerdione-[3], dehydrogingerdione-[4]) (in particular, those as described in WO 2007/003527, which, with respect to the corresponding compounds disclosed therein, becomes part of this application by way of reference); diacetyl trimers (in particular those as described in WO 2006/058893, which, with respect to the corresponding compounds disclosed therein, becomes part of this application by way of reference); gamma-amino butyric acids (in particular those as described in WO 2005/096841, which, with respect to the corresponding compounds disclosed therein, becomes part of this application by way of reference); divanillins (in particular those as described in WO 2004/078302, which, with respect to the corresponding compounds disclosed therein, becomes part of this application by way of reference); 4-hydroxydihydrochalcones (in particular, those as described in US 2008 0227867, which, with respect to the corresponding compounds disclosed therein, becomes part of this application by way of reference); in this context, in particular, phloretin and davidigenin; amino acids or mixtures of whey proteins with lecithins, yeast extracts, plant hydrolysates, powdered vegetables (e.g. onion powder, tomato powder), plant extracts (e.g. of lovage or of mushrooms such as shiitake), seaweed and mineral salt mixtures and mixtures (in particular those as described in WO 2007/045566, which, with respect to the compounds disclosed therein, becomes part of this application by way of reference); hesperetin (in particular those as disclosed in WO 2007/014879, which, with respect to the corresponding compounds disclosed therein, becomes part of this application by way of reference); 4-hydrochalcones (in particular those as disclosed in WO 2007/107596, which, with respect to the corresponding compounds disclosed therein, becomes part of this application by way of reference); propylene phenyl glycosides (chavicol glycosides) (in particular those as described in EP 1 955 601, which, with respect to the corresponding compounds disclosed therein, becomes part of this application by way of reference); Pellitorin and aroma compositions derived therefrom; Umami compounds (in particular those as described in WO 2008/046895 and EP 1 989 944, which, with respect to the compounds disclosed therein, become part of this application by way of reference); Matairesinol and neoflavanoids (particularly those described in WO 2012/146584, US 2013 078192 and US 2013 084252, which become part of this application by way of reference with respect to the compounds disclosed therein); and neoisoflavonoids (particularly those described in EP 2 570 035, EP 2 570 036 and EP 2 726 026, which become part of this application by way of reference with respect to the compounds disclosed therein), extracts of Mycetia ssp. or balansins as described in EP 2 529 633 B1, hesperetindihydrochalcone as described in WO 2017 186,299, mixtures containing naringenin and phloretin as described in WO 2017 092,796, native or transglycosidated rubusosides as in EP 2 386 211 B1 or WO 2015 189,346, flavans as described in U.S. Pat. No. 2,010,292,175, phyllodulcin or hydrangea extracts as described in EP 2,298,084, dihydrochaklcones as described in EP 2,353,403, homovanillic acid esters as described in EP2,932,858, phyllodulcin degradation products as described in PCT/EP2017/065457.

Bicyclo[4.1.0]heptane-7-carboxamides, in particular those described in EP 2 079 322 (Symrise), which, with respect to the corresponding compounds disclosed therein, becomes part of this application by way of reference; cyclopropanecarboxylic acid (3-methylcyclohexyl) amides, in particular those described in EP 1 989 944 (Symrise), which, with respect to the corresponding compounds disclosed therein, becomes part of this application by way of reference; aromatic neo-menthylamides, in particular those described in EP 2 064 959 (Symrise), which, with respect to the corresponding compounds disclosed therein, becomes part of this application by way of reference; neo-menthylamides, in particular those described in US 2009 311401 (Symrise), which, with respect to the corresponding compounds disclosed therein, becomes part of this application by way of reference; geranylamine derivatives of oxalic acid as well as neomenthyl derivatives.

Generally, the two, three, more or all of the further flavoring agents for imparting, modifying and/or enhancing one, two or all of the taste sensations umami, kokumi and salty (taste modulators) are preferably naturally occurring compounds, particularly preferably compounds selected from the group consisting of: Monosodium glutamate, free glutamic acid, nucleotides such as adenosine 5′-monophosphate, cytidine 5′-monophosphate, inosine 5′-monophosphate, guanosine 5′-monophosphate) and pharmaceutically acceptable salts thereof; Strombins (in particular, those as described in WO 2010/100589, which, with respect to the corresponding compounds disclosed therein, becomes part of this application by way of reference); theogallins (in particular, those as described in JP 2007 110988, which, with respect to the corresponding compounds disclosed therein, becomes part of this application by way of reference); pyridine-betaine compounds (in particular, those as described in EP 1 291 342, which, with respect to the corresponding compounds disclosed therein, becomes part of this application by way of reference); glutamic acid glycosides (in particular, those as described in WO 2002/087361, which, with respect to the corresponding compounds disclosed therein, becomes part of this application by way of reference); malic acid glycosides (in particular, those as described in WO 2006/003107, which, with respect to the corresponding compounds disclosed therein, becomes part of this application by way of reference); glutathione derivatives (in particular those as described in EP 0 181 421 or WO 2007/042273, which, with respect to the corresponding compounds disclosed therein, become part of this application by way of reference); lactisols and alkylpyridines (in particular such alkylpyridines as described in WO 2009/122318 and WO 2009/1223319, which, with respect to the corresponding compounds disclosed therein, become part of this application by way of reference), in particular 2-hexyl-, 2-heptyl- and 2-octylpyridine, (2E,6Z)-N-cyclopropylnona-2,6-dienamide, (2E,6Z)—N-ethylnona-2,6-dienamide, N-[(2E)-3,7-dimethylocta-2,6-dienyl]-cyclopropanecarboxamide, N′-[(2-methoxy-4-methyl-phenyl)methyl]-N-[2-(5-methyl-2-pyridyl)ethyl]-oxamide, N′-[(2,4-dimethoxyphenyl)methyl]-N-[2-(2-pyridyl)ethyl]-oxamide, N′-[(2-methoxy-4-methyl-phenyl)-methyl]-N-[2-(2-pyridyl)-ethyl]-oxamide, N-(1-propylbutyl)-1,3-benzodioxole-5-carboxamide, 1-(2-hydroxy-4-isobutoxy-phenyl)-3-(2-pyridyl)-propan-1-one and 1-(2-hydroxy-4-methoxy-phenyl)-3-(2-pyridyl)-propan-1-one; cinnamic acid amides, in particular rubemamide or rubescenamide; hydroxyflavones, such as eriodictyol, homeriodictyol or their sodium salts); hesperetin, pholoeretin, hydroxyflavans, 4-hydroxychalcones; extracts based on Hydrangea dulcis (in particular those as described in EP 2 298 084, which in this respect becomes part of this application by way of referral) or Rubus ssp. mixtures of whey proteins with lecithins, yeast extracts, plant hydrolysates, powdered vegetables (e.g. onion powder, tomato powder), plant extracts (e.g. of lovage or of mushrooms such as shiitake), seaweed and mineral salt mixtures (in particular mineral salt mixtures as described in US 2009 214728, which in this respect becomes part of this application by way of reference).

Synthetic flavoring agents preferably to be used in the combination agent according to the invention for imparting, modifying and/or enhancing one, two or all of the taste sensations umami, kokumi and salty are preferably selected from the chemical substances, described in publications US 2004 0202619, US 2004 0202760, US 2006 0057268 and US 2006 0068071, in particular (2E,6Z)—N-cyclopropylnona-2,6-dienamide, (2E,6Z)—N-ethylnona-2,6-dienamide and N-[(2E)-3,7-dimethylocta-2,6-dienyl]-cyclopropanecarboxamide, the chemical structures as described in US 2005 0084506, in particular N′-[(2-methoxy-4-methylphenyl)-methyl]-N-[2-(5-methyl-2-pyridyl)ethyl]-oxamide, N′-[(2,4-dimethoxyphenyl)-methyl]-N-[2-(2-pyridyl)-ethyl]-oxamide, N′-[(2-methoxy-4-methylphenyl)-methyl]-N-[2-(2-pyridyl)ethyl]-oxamide, N-(1-propylbutyl)-1,3-benzodioxole-5-carboxamide, and the chemical structures as described in WO 2011/004016, in particular 1-(2-hydroxy-4-isobutoxyphenyl)-3-(2-pyridyl)-propan-1-one and 1-(2-hydroxy-4-methoxyphenyl)-3-(2-pyridyl)-propan-1-one; rubemamine, rubemamide, rebuscenamine, rubescenamide, zanthosine, zanthosinamide, dioxamine, dioxamide, zanthomamine and zanthomamide and mixtures thereof as described in EP 2 529 632 or EP 2 737 807.

In addition to one or more sweet-enhancing substance(s) or to one or more flavor modulator(s), the combination agent according to the invention may preferably contain flavoring substances that provide a tingling (tingling) or cooling effect. Such flavoring agents are preferably selected from the group consisting of: Menthol and menthol derivatives such as, for example, L-menthol, D-menthol, racemic menthol, isomenthol, neoisomenthol, neomenthol; menthyl ethers such as, for example, 3-(I-menthoxy)-1,2-propanediol, 3-(I-menthoxy)-2-methyl-1,2-propanediol, 3-(I-menthyl methyl ether; Menthone glyceryl acetal, menthone glyceryl ketal or mixtures thereof; menthyl esters such as, for example, menthyl formate, menthyl acetate, menthyl isobutyrate, menthydroxy isobutyrate, menthyl lactate, L-menthyl L-lactate, L-menthyl D-lactate, menthyl (2-methoxy)acetate, menthyl (2-methoxyethoxy)acetate, menthyl pyroglutamate; menthyl carbonates such as menthyl propylene glycol carbonate, menthyl ethylene glycol carbonate, menthyl glycerol carbonate or mixtures thereof; the half esters of menthols with a dicarboxylic acid or derivatives thereof such as, for example, monomenthyl succinate, monomenthyl glutarate, monomenthyl malonate, O-menthyl succinic acid ester N,N-(dimethyl) amide, O-menthyl succinic acid ester amide), Menthanecarboxylic acid amides (preferably menthanecarboxylic acid-N-ethylamide or N^{α}-(menthanecarboxylic acid) glycine ethyl ester as described in U.S. Pat. No. 4,150,052, menthanecarboxylic acid-N-(4-cyanophenyl)-amide or menthanecarboxylic acid-N-(4-cyanomethylphenyl)-amide as described WO 2005/049553); menthanecarboxylic acid-N-(alkoxyalkyl)-amide); menthone and menthone derivatives such as for example L-menthone glycerol ketal); 2,3-dimethyl-2-(2-propyl)-butyric acid derivatives such as for example 2,3-dimethyl-2-(2-propyl)-butyric acid-N-methylamide, isopulegol or its esters such as for example (I-(−)-isopulegol, I-(−)-isopulegol acetate; Menthane derivatives such as p-menthane-3,8-diol; cubebol or synthetic or natural mixtures containing cubebol; Pyrrolidone derivatives of cycloalkyldione derivatives such as for example 3-methyl-2 (1-pyrrolidinyl)-2-cyclopentene-1-one or tetrahydropyrimidine-2-ones such as for example icilin or related compounds as described in WO 2004/026840); Carboxamides such as for example N-(2-(pyridin-2-yl)-ethyl)-3-p-menthanecarboxamide or related compounds such as for example (1R,2S,5R)—N-(4-methoxyphenyl)-5-methyl-2-(1-isopropyl) cyclohexanecarboxamides; Oxamates, preferably those described in EP 2 033 688 and [(1R,2S,5R)-2-isopropyl-5-methyl-cyclohexyl] 2-(ethylamino)-2-oxo-acetate (X Cool).

Optional Ingredient (e)—Carrier and/or Excipient or Adjuvant:

In another embodiment, the combination agent according to the first aspect of the present invention may comprise, as a further component, at least one excipient and/or pharmaceutically active adjuvant suitable for consumption.

The at least one carrier to be used in the combination agent according to the invention is preferably selected from the group consisting of: Silicon dioxide (silicic acid, silica gel); Carbohydrates and/or carbohydrate polymers (polysaccharides); Dietary fibers insoluble in particular under physiological conditions, in particular selected from the group consisting of: Cellulose, modified cellulose, hemicelluloses, lichenin, chitin, chitosan, lignins, xanthan gum, vegetable fibers, in particular cereal fibers, potato fibers, apple fibers, citrus fibers, bamboo fibers, extracted sugar beet fibers, oat fibers; dietary fibers soluble under physiological conditions, in particular selected from the group consisting of: Inulin, especially native inulin, highly soluble inulin, granulated inulin, high performance inulin, pectins, alginates, agar, carrageenan, gum arabic, guar gum, locust bean gum, xanthan gum, raffinose, xylose, polydextrose, lactulose, gum ghatti, traganth; Flours, especially flours from wheat (soft or durum wheat), rye, oats, barley, emmer, spelt, einkorn, buckwheat, millet, corn, rice; Starch, in particular starch from wheat, potatoes, corn, rice, tapioca and oats, chemically or physically modified starch, degraded starch (starch hydrolysates) and starch derivatives, preferably dextrins or maltodextrins, in particular dextrins and maltodextrins from wheat, potatoes, corn, rice and oats, cyclodextrins, oligosaccharides, in particular oligofructose; sugar alcohols, in particular sorbitol, mannitol, isomalt, maltitol, maltilol syrup, lactitol, xylitol, erythritol; and mixtures of the aforementioned carriers.

Dietary fibers are largely indigestible food components. Dietary fiber increases the volume of food simply by its presence, without also increasing the energy content at the same time. Some dietary fibers have the property of binding water, which causes them to absorb water in the stomach. The resulting mechanical increase in volume leads to greater distension of the gastric sac, which in turn leads to a reduction in the appetite-stimulating ghrelin level and thus to an increase in the feeling of satiety. The addition of a dietary fiber to the combination agent according to the invention can thus additionally reduce appetite.

Preferred carriers are silicon dioxide, gum arabic and maltodextrins, with maltodextrins with DE values in the range 5 to 20 again being preferred. It is irrelevant which plant originally supplied the starch for the production of the starch hydrolysates. Suitable and readily available are corn-based starches and starches from tapioca, rice, wheat or potatoes.

According to the invention, the above-mentioned carriers for preparing the combination agent according to the invention are used either individually or in combination with one another as a mixture.

The at least one excipient to be used in the combination agent according to the invention, in particular pharmaceutical excipient, is selected, depending on the dosage form of the combination agent according to the invention, from the group consisting of: Fillers, solvents, humectants, emulsifiers, solubilizers, wetting agents, buffers, thickeners and binders, coating agents, disintegration accelerators, disintegrating agents, lubricants, mold release agents, flow regulators, antioxidants, preservatives and absorption accelerators.

Such excipients are substances used in the preparation of the combination agent, in particular in the preparation of medicinal products. Such excipients are optimally pharmacologically and toxicologically inert and have the following functions:
Shaping: excipients carry the drug substance and give the drug its shape;
Manufacturability: excipients enable or improve certain manufacturing steps in drug production;
Control of drug release: excipients specifically control the release of active ingredients or specifically transport them to their site of action; and
Stability enhancement: excipients ensure that a drug has a sufficient shelf life.

According to the invention, the above-mentioned excipients for the preparation of the combination agent according to the invention are used either individually or in combination with each other as a mixture.

In a further preferred embodiment of the first subject matter of the invention, the combination means according to the invention is characterized in that it comprises or consists of:
0.00001 (0.1 ppm) to 0.001 (10 ppm) wt % of the one or more first saturation component(s);
0.1 to 20% by weight, preferably 5 to 20% by weight, of added free arginine, in particular free L-arginine; and
optionally
1 to 80% by weight, preferably 1 to 95% by weight, of the one or more second saturation component(s); and/or
0.00001 (0.1 ppm) to 1% by weight of the one or more sensory active substance; in each case based on the total weight of the combination agent.

Particularly advantageous are compositions of the combination agent in which the proportion of added free arginine in the combination agent is at least 0.5 to 10% by weight, in particular the content of added free L-arginine is at least 0.5 to 10% by weight, based on the total weight of the combination agent.

The combination agent according to the invention, in particular according to the above variants, is characterized by the fact that it reduces the feeling of hunger or acts against cravings, provides a feeling of satiety and/or stimulates the metabolism.

The effect of the combination agents according to the invention was tested on 32 subjects:

TABLE 2
Information on the test subjects

| Variable | Mean value | Standard deviation | Area |
|---|---|---|---|
| Age (years) | 25.8 | 3.6 | 20-36 |
| Size (m) | 1.82 | 0.1 | 1.68-1.93 |
| Weight (kg) | 89.3 | 11.2 | 69.5-111 |
| BMI (kg/m2) | 26.8 | 1.9 | 24.0-31.9 |
| Blood glucose after | 74.6 | 7.3 | 62-97 |
| fasting (mg/dl) | | | |

For testing, 32 subjects underwent a medical examination and received test solutions within an oral glucose tolerance test (OGTT) as a stimulant for satiety on four different days. On the test days, after an overnight fast of 12 hours, subjects rated their subjective feeling of hunger on a Visual Analog Scale (VAS) (scale measuring a subjective attitude) and then received either:
C (control): 75 g glucose plus 0.15 mg nonivamide in 300 ml water (first saturation component);
ARG: 75 g glucose plus 0.15 mg nonivamide plus 1.2 g L-arginine in in 300 ml water (combination agent according to the present invention);
WPH: 75 g glucose plus 0.15 mg nonivamide plus 2 g wheat protein hydrolysate (argine content: about 2 to 3%) in 300 ml water (second saturation component), or.
WPH+ARG: 75 g glucose plus 0.15 mg nonivamide plus 2 g wheat protein hydrolysate plus 1.2 g L-arginine in 300 ml water (combination agent according to the present invention).

The nonivamide glucose drink also contained 150 mg of ¹³C—Na-acetate in 300 ml of water and 15 μl of ethanol.

In parallel, breath samples were collected from the subjects as the test solutions were labeled with ¹³C—Na-acetate, a marker to detect gastric emptying. Within the next 140 minutes, 6 blood samples and 11 breath samples were collected and analyzed from each subject. Before the subjects consumed a standardized ad libitum breakfast to analyze food intake, subjects again rated their subjective feeling of hunger on a Visual Analog Scale (VAS).

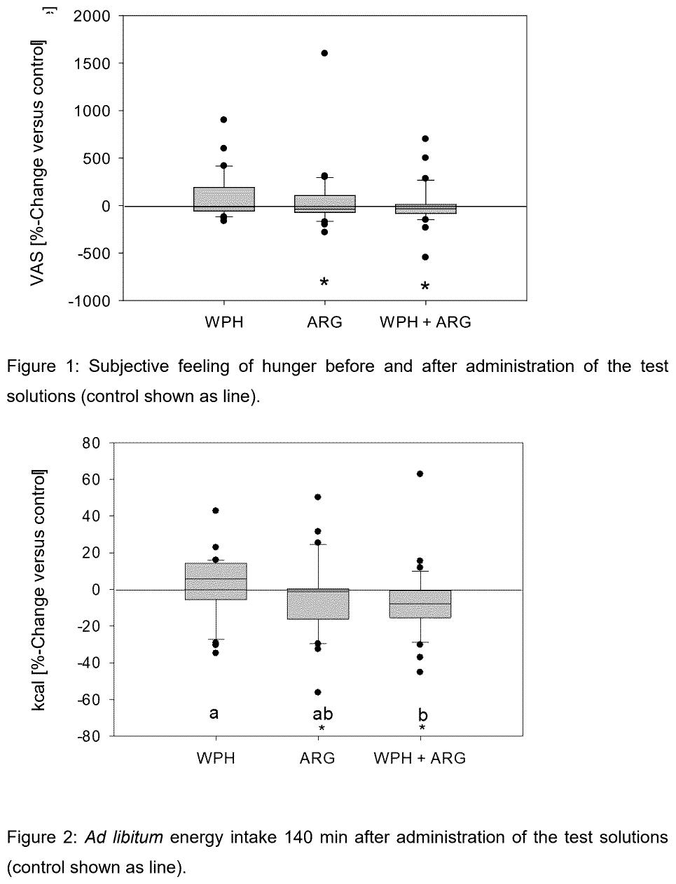

The test results are shown in FIGS. 1 to 4.

As can be seen from FIG. 1, the feeling of hunger after administration of the combination agents according to the invention (ARG and WPH+ARG) was significantly lower than in the control (shown as a line) and the composition WPH not according to the invention. The feeling of hunger was at least pronounced after administration of the combination agent (WPH+ARG) according to the invention.

As can be seen from FIG. 2, the ad libitum energy intake 140 min after administration of the combination agents according to the invention (ARG and WPH+ARG) was significantly lower than that of the control (shown as a line) and the composition WPH not according to the invention. Administration of the combination agent (WPH+ARG) according to the invention resulted in the ad libitum energy intake being the lowest.

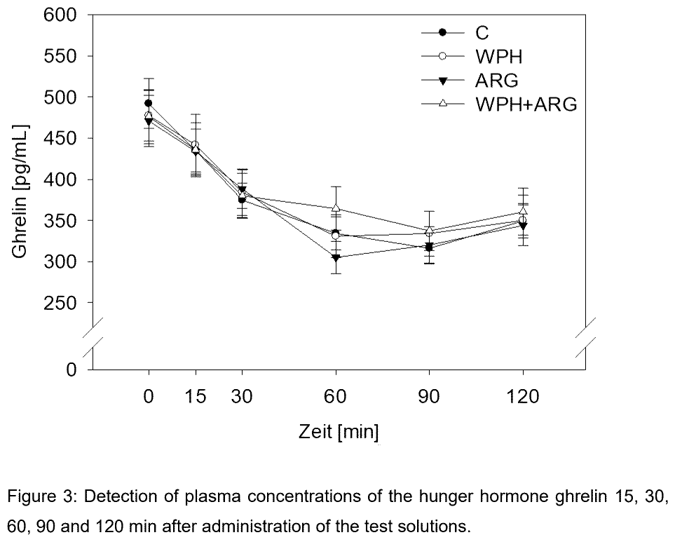

As can be seen from FIG. 3, the combination agent (WPH+ARG) according to the invention has no effect on the hunger hormone ghrelin.

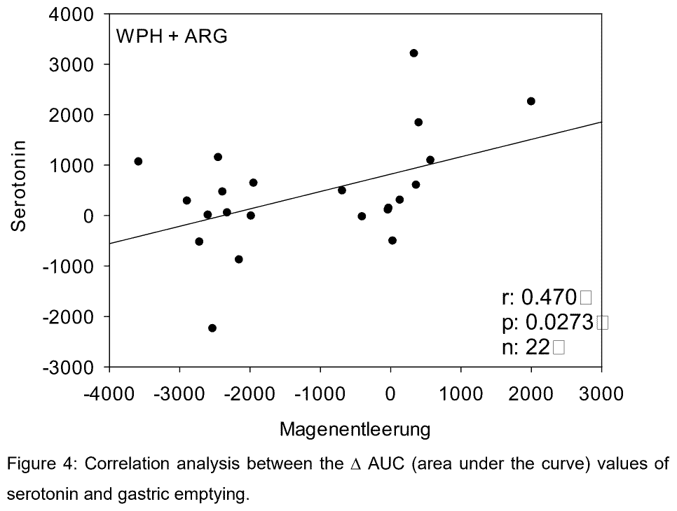

In FIG. 4, a correlation analysis shows that serotonin correlates with gastric emptying after administration of the combination agent of the invention (WPH+ARG), so that an increase in satiety can be assumed.

As a plasma glucose level study showed, the glucose level in plasma after administration of the combination agents according to the invention (ARG and WPH+ARG) is significantly lower than for the control and the composition WPH not according to the invention. The best results were obtained for the combination agent that contained a second saturating component (WPH+ARG).

TABLE 3

| Time | Control | WPH | ARG | WPH + ARG |
|---|---|---|---|---|
| [min] | Δ [mg/dL] | Δ [mg/dL] | Δ [mg/dL] | Δ [mg/dL] |
| 0 | 0 | 0 | 0 | 0 |
| 15 | 28.42 ± 3.62 | 43.65 ± 4.20 | 34.14 ± 6.51 | 25.78 ± 3.90 |
| 30 | 44.62 ± 4.42 | 55.08 ± 5.01 | 42.21 ± 5.11 | 53.80 ± 5.57 |
| 60 | 20.15 ± 7.47 | 24.48 ± 6.66 | 17.56 ± 9.40 | 25.34 ± 6.59 |
| 90 | 5.37 ± 6.58 | 6.50 ± 6.03 | −2.80 ± 6.28 | 0.98 ± 4.31 |
| 120 | −15.40 ± 5.11 | −6.46 ± 4.40 | −18.66 ± 4.46 | −10.81 ± 4.77 |

From the above values, it can be concluded that regular use of the combination agent according to the invention can reduce the increase in the glycemic index and its negative metabolic consequences.

TABLE 4
Detection of ¹³CO₂ in breath as a marker for gastric emptying.

| | Δ Change in | | |
|---|---|---|---|
| | gastric emptying | t-test vs. | Mechanical |
| | compared to control | control p- | saturation |
| Treatment | [Treatment − Control] | value | signal |
| WPH* | −791.7 ± 367.6 | 0.04 | ↑ increases |
| | | | significantly |
| ARG | −634.4 ± 398.2 | 0.12 | =no change |
| WPH + ARG* | −1129.0 ± 323.9 | 0.001 | ↑ increases |
| | | | significantly |

The principle of this test is based on the fact that the test solutions were labeled with ¹³C—Na-acetate. ¹³C—Na-acetate is not absorbed in the stomach, but only in the small intestine. Metabolization occurs in the liver, so that ¹³CO₂ is subsequently exhaled through the lungs. The ¹³CO₂ concentration can be used to determine whether the test solutions have already left the stomach. The higher the concentration of ¹³CO₂ in the breath, the faster the gastric emptying. For the calculation, the basal ¹²CO₂ value as well as the body surface area was taken into account by means of the DuBois formula of each test person.

TABLE 5
Detection of the satiety hormone serotonin after administration
of wheat protein alone and in combination with arginine.

| | Δ Change of serotonin | t-test vs. | Hormonal |
|---|---|---|---|
| | compared to control | control p- | Saturation |
| Treatment | [Treatment − Control] | value | signal |
| WPH | −28.1 ± 154.9 | 0.813 | — |
| ARG | 283.9 ± 189.8 | 0.222 | — |
| WPH + ARG* | 350.4 ± 218.2 | 0.007 | ↑ increases |
| | | | significantly |

As the above studies clearly demonstrate, the administration of a combination agent according to the invention leads to a reduction in appetite and to the imparting of a more rapid feeling of satiety, and consequently to a reduction in energy intake

The combination agent according to the invention, which in addition to the ingredients (a) to (d) to be used according to the invention may also comprise one or more carriers or auxiliaries, may be prepared, for example, by mechanical mixing processes, in which the particles may also be comminuted at the same time, or by means of spray drying. Preferably, a combination agent according to the invention comprises solid carriers or excipients and is prepared by means of spray drying. Alternatively, both the one or more first saturating component(s) (ingredient (a)) and the one or more added free amino acid(s) (ingredient (b)) may be prepared as solutions and added to the one or more second saturating component(s) (ingredient (c)). The flavoring agents (ingredient (d)) may be used as such or in a stand-alone preparation well known to those skilled in the art, either as a solution or as a carrier.

Preferred combination agents comprising carriers according to the invention and prepared by spray drying have an average particle size in the range of 3 to 1000 μm and a residual moisture content of less than or equal to 5% by weight.

A second object of the invention and aspect relates to the combination agent according to the invention for medical use, i.e. for use in a therapeutic procedure, in particular as an agent for reducing appetite and/or as an agent for imparting a feeling of satiety, and concomitantly for reducing caloric intake. The combination agent according to the invention is therefore preferably a medicament or is used for use as a medicament.

In the therapeutic method, the combination agent according to the invention is used in such a way that it reduces appetite and/or induces a feeling of satiety and thus leads to a reduction in caloric intake. Preferably, according to a variant of the second aspect, the combination agent is therefore used for treating overweight or obesity.

Due to the reduction in caloric intake resulting from a reduced appetite and a faster feeling of satiety, the combination agent according to the invention is particularly suitable for the therapeutic reduction of weight and thus for the therapeutic treatment of overweight and obesity. Obesity is a nutritional and metabolic disease with severe overweight, which is characterized by an increase in body fat above the normal level.

Scientific studies have shown that obesity is directly related to cardiovascular disease, stroke, diabetes, and joint damage and wear and tear of the spine.

By reducing caloric intake and thus reducing body weight, the combination agent according to the invention is also advantageously suitable for the prevention of cardiovascular diseases, in particular hypertension, diabetes, in particular type 2 diabetes mellitus, stroke and joint damage.

In another preferred embodiment, the combination agent is used to prevent cardiovascular disease, stroke, diabetes, and joint damage and wear of the spine.

The second subject matter and aspect of the invention further relates to the combination agent according to the invention for non-therapeutic use, in particular as an agent for reducing appetite and/or as an agent for providing a feeling of satiety, and concomitantly for reducing caloric intake.

In the non-therapeutic method, the combination agent according to the invention is used in such a way that it reduces appetite and/or induces a feeling of satiety and thus leads to reduced caloric intake.

By reducing caloric intake, the combination agent according to the invention is advantageously suitable for cosmetic weight reduction or in prophylaxis against obesity.

In a most preferred embodiment according to another aspect of the present invention, the combination agent is a dietary supplement or a drug. The dietary supplement or medicament may be in the form of powders, solid mixtures, granules, pellets, capsules, tablets (uncoated as well as coated tablets), coated tablets, solutions, dispersions in liquid phases, emulsions, pastes, or as other preparations that can be swallowed or chewed.

The third subject matter and aspect of the present invention relates to the use of the combination agent of the invention as a means for
(i) Reduction of appetite; and/or
(ii) Providing a feeling of satiety; and/or
(iii) Reduction of energy intake; and/or
(iv) Reduction of body weight.

In order to support a reduction in body weight in a patient or consumer, it has been found useful to limit caloric intake. This can be done by reducing appetite and providing a quicker feeling of satiety with oral ingestion of the combination agent of the invention. As a result, a patient or consumer consumes less, thereby reducing energy intake.

As demonstrated by the studies described above, the combination agent according to the invention can be used to reduce appetite and/or provide a faster feeling of satiety. Due to these properties, the combination agent is excellently suited as a means of reducing energy intake and/or thus reducing body weight.

According to a further aspect, the present invention relates to the use of the combination agent according to the invention for the preparation of an orally consumable preparation or food ingredient.

Orally consumable preparations, in particular foodstuffs, food supplements, feedstuffs or medicinal products, are any preparations or compositions suitable for consumption for the purpose of nutrition, oral hygiene or enjoyment and are regularly products which are intended to be introduced into the human or animal oral cavity for a certain period of time and then either to be consumed (e.g. ready-to-eat foodstuffs or feedstuffs) or to be removed from the oral cavity (e.g. chewing gum). These products include all substances or products that are intended to be ingested by humans or animals in a processed, partially processed, or unprocessed state. This also includes substances that are added to orally consumable products (in particular foodstuffs, feedstuffs and medicinal products) during their manufacture, processing or treatment and are intended to be introduced into the human or animal oral cavity.

This includes in particular (calorie-reduced) bakery products (e.g. bread, dry cookies, cakes, other pastries), confectionery (e.g. chocolates, chocolate bar products, other bar products, fruit gums, coated tablets, hard and soft caramels, chewing gum), non-alcoholic beverages (e.g. Cocoa, coffee, green tea, black tea, (green, black) tea beverages fortified with (green, black) tea extracts, rooibos tea, other herbal teas, fruit-flavored sodas, isotonic beverages, soft drinks, nectars, fruit and vegetable juices, fruit or vegetable juice preparations), instant beverages (e.g. instant cocoa drinks, instant tea drinks, instant coffee drinks), meat products (e.g. ham, fresh or raw sausage preparations, seasoned or marinated fresh or cured meat products), insects or insect products, eggs or egg products (dried egg, egg white, egg yolk), cereal products (e.g., breakfast cereals, granola bars, pre-cooked ready-to-eat rice products), dairy products (e.g. Full-fat or reduced-fat or fat-free but preferably high-protein dairy drinks, rice pudding, yogurt, kefir, cream cheese, soft cheese, hard cheese, dried milk powder, whey, butter, buttermilk, partially or fully hydrolyzed milk protein-containing products), products high in vegan, preferably plant-based proteins, e.g. Algae, wheat, rapeseed, sunflower, rice, potato, corn, soybean, bean, pea, lentil, lupin, peanut, alfalfa protein isolates, protein fractions, partial or complete protein hydrolysates, or physico-chemically, enzymatically or fermentatively modified proteins of the aforementioned sources, e.g. from soy protein or other soybean fractions (e.g. Soy milk and products made therefrom, beverages containing isolated or enzymatically treated soy protein, beverages containing soy flour, preparations containing soy lecithin, fermented products such as tofu or tempeh or products made therefrom, and mixtures with fruit preparations and optional flavorings), meat substitutes made from the aforementioned vegetarian or vegan protein sources, fruit preparations (e.g. jams, fruit ice cream, fruit sauces, fruit fillings), vegetable preparations (e.g., ketchup, sauces, dried vegetables, frozen vegetables, pre-cooked vegetables, canned vegetables), snack foods (e.g., baked or fried potato chips or potato dough products, corn- or peanut-based extrudates), fat- and oil-based products or emulsions thereof (e.g. mayonnaise, tartar sauce, dressings, in each case full-fat or reduced-fat), other ready-to-serve meals and soups (e.g. dry soups, instant soups, pre-cooked soups), spices, seasoning mixtures and, in particular, seasonings which are used, for example, in the snack sector, sweetener preparations, tablets or sachets, other preparations for sweetening or whitening beverages or other foodstuffs. The preparations within the meaning of the invention may also serve as semi-finished products for the production of other preparations for nutrition or enjoyment.

Particularly preferred are calorie-reduced confectionery (e.g., cereal bar products, fruit gums, dragées, hard and soft caramels, chewing gum), non-alcoholic beverages (e.g. Cocoa, green tea, black tea, (green, black) tea beverages fortified with (green, black) tea extracts, rooibos tea, other herbal teas, fruit-flavored sodas, isotonic beverages, soft drinks, nectars, fruit and vegetable juices, fruit or vegetable juice preparations), instant beverages (e.g. instant cocoa drinks, instant tea drinks), cereal products (e.g., breakfast cereals, granola bars, pre-cooked ready-to-eat rice products), dairy products (e.g., full-fat or reduced-fat or fat-free dairy drinks, rice pudding, yogurt, kefir, dry milk powder, whey, buttermilk, partially or wholly hydrolyzed milk protein-containing products), products high in vegan, preferably plant-based proteins, e.g. algae, wheat, rapeseed, sunflower, rice, potato, corn, soy, bean, pea, lentil, lupin, peanut, alfalfa protein isolates, protein fractions, partial or complete protein hydrolysates, or physico-chemically, enzymatically or fermentatively modified proteins of the aforementioned sources, products made from soy protein or other soybean fractions (e.g. Soy milk and products made therefrom, beverages containing isolated or enzymatically treated soy protein, beverages containing soy flour, preparations containing soy lecithin, fermented products such as tofu or tempe or products made therefrom, and mixtures with fruit preparations and optional flavorings), sweetener preparations, tablets or sachets, other preparations for sweetening or whitening beverages or other foodstuffs, vegetable preparations (e.g., ketchup, sauces, dried vegetables, frozen vegetables, pre-cooked vegetables, canned vegetables), prepared meals and soups (e.g., dried soups, instant soups, precooked soups) Meat substitutes made from aforementioned vegetarian or vegan protein sources.

In the context of the present invention, a food ingredient is to be understood as an orally consumable substance or an orally consumable product which is used as a constituent and, where appropriate, with the application of further process steps, for the production of a ready-to-use or ready-to-eat preparation (in particular a foodstuff, a food supplement or a feedstuff) which is used for nutrition or enjoyment.

Other components of an orally consumable preparation for nutrition or enjoyment can be common basic substances, auxiliary substances and additives for foodstuffs or semi-luxury foods, e.g. water, mixtures of fresh or processed, vegetable or animal basic substances or raw materials (e.g. raw, roasted, dried, fermented, smoked and/or cooked meat, bones, cartilage, fish, vegetables, herbs, nuts, vegetable juices or pastes or mixtures thereof), digestible or non-digestible carbohydrates (e.g. Sucrose, maltose, fructose, glucose, dextrins, amylose, amylopectin, inulin, xylans, cellulose, tagatose), sugar alcohols (e.g., sorbitol, erythritol), natural or hydrogenated fats (e.g., tallow, lard, palm fat, coconut fat, hydrogenated vegetable fat), oils (e.g. Sunflower oil, peanut oil, corn oil, olive oil, fish oil, soybean oil, sesame oil), fatty acids or their salts (e.g., potassium stearate), proteinogenic or non-proteinogenic amino acids and related compounds (e.g., γ-aminobutyric acid, taurine), peptides (e.g., glutathione), native or processed proteins (e.g., gelatin), enzymes (e.g. peptidases), nucleic acids, nucleotides, taste correctors for unpleasant taste sensations, other taste modulators for further, usually not unpleasant taste sensations, other taste modulating substances (e.g. inositol phosphate, nucleotides such as guanosine monophosphate, adenosine monophosphate or other substances such as sodium glutamate or 2-phen-oxypropionic acid), emulsifiers (e.g. lecithins, diacylglycerols, gum arabic), stabilizers (e.g. carrageenan, alginate), preservatives (e.g. benzoic acid and its salts, sorbic acid and its salts), antioxidants (e.g. tocopherol, ascorbic acid), chelators (e.g. citric acid), organic or inorganic acidifiers (e.g. acetic acid, phosphoric acid), additional bitter substances (e.g. quinine, caffeine, limonine, amarogentin, humolones, lupolones, catechins, tannins), substances that prevent enzymatic browning (e.g. sulfite, ascorbic acid), essential oils, plant extracts, natural or synthetic colorants or color pigments (e.g. carotenoids, flavonoids, anthocyanins, chlorophyll and their derivatives), spices, trigeminally active substances or plant extracts containing such trigeminally active substances, synthetic, natural or nature-identical flavoring substances or fragrances, and odor-correcting agents.

The energy density of an orally consumable product (in particular a food, a food supplement, a feed or a drug) can be reduced by replacing energy-rich ingredients of the orally consumable product with substitutes (e.g. low-calorie thickeners instead of fats, low-calorie or calorie-free sweeteners instead of common sugars).

In addition, the energy density of an orally consumable product can be further reduced by dietary fiber. Dietary fibers are largely indigestible food components. Dietary fiber increases the volume of food simply by its presence, without also increasing the energy content at the same time. Some dietary fibers have the property of binding water, which causes them to absorb water in the stomach. The resulting mechanical increase in volume leads to greater distension of the gastric sac, which in turn leads to a reduction in the appetite-stimulating ghrelin level and thus to an increase in the feeling of satiety. Thus, appetite can be reduced at the same time. Preferably, an orally consumable product to be used according to the invention is selected from the group consisting of confectionery, preferably reduced-calorie or calorie-free confectionery, preferably selected from the group consisting of cereal bar products, fruit gums, coated tablets, hard caramels and chewing gum,
non-alcoholic beverages, preferably selected from the group consisting of green tea, black tea, (green, black) tea beverages fortified with (green, black) tea extracts, rooibos tea, other herbal teas, fruit-containing low-sugar or sugar-free lemonades, isotonic beverages, nectars, fruit and vegetable juices, fruit and vegetable juice preparations,
Instant beverages, preferably selected from the group consisting of instant (green, black, rooibos, herbal) tea beverages,
Cereal products, preferably selected from the group consisting of low and no sugar breakfast cereals and cereal bars,
Dairy products, preferably selected from the group consisting of reduced-fat and non-fat dairy beverages, yogurt, kefir, whey, buttermilk and ice cream,
Products made from soy protein or other soybean fractions, preferably selected from the group consisting of soy milk, products made from soy milk, beverages containing isolated or enzymatically treated soy protein, beverages containing soy flour, preparations containing soy lecithin, products made from preparations containing soy lecithin, and mixtures with fruit preparations and, optionally, flavors,
products from other vegetable proteins or protein fractions, wherein the source of the proteins is preferably selected from the group consisting of sweet lupin, pea, bean, lentil, wheat, canola, algae, potato and rice,
Sweetener preparations, tablets and sachets
sugar-free dragées,
Ice cream, with or without ingredients based on milk, preferably sugar-free.
savory (“savory”) foods, preferably selected from the group consisting of meat substitutes, pasta dishes, soups, protein-rich soups, etc.

According to a further aspect, the present invention relates to an orally consumable preparation or food ingredient comprising the combination agent according to the invention.

In a preferred embodiment, the orally consumable preparation or food ingredient according to the invention comprises the combination agent according to the invention in combination with one or more dietary fiber(s).

In such an orally consumable preparation or foodstuff, the combination agent is present in an amount of 1 to 80% by weight, preferably 3 to 50%, particularly preferably 5 to 30%, in each case based on the total weight of the orally consumable preparation.

### DESIGN EXAMPLES

### Application Example 1: Low-Fat Yogurts

For the production of yogurts, the ingredients are mixed according to the recipes mentioned below and cooled to 5° C.

| | Preparation (data as % by weight) | | | |
|---|---|---|---|---|
| Ingredient | A | B | C | D |
| Sucrose | 10 | 8 | 6 | — |
| Rebaudioside A 98% | — | — | — | 0.050 |
| Extract of Rubus suavissimus | — | 0.010 | 0.010 | — |
| containing 20% by weight of | | | | |
| rubusoside, based on the total | | | | |
| weight of the extract, e.g. of | | | | |
| PlantExtract | | | | |
| Strawberry flavor | 0.1 | 0.1 | 0.1 | 0.1 |
| Hesperetin | — | 0.001 | 0.001 | 0.002 |
| Phloretin | — | — | 0.002 | 0.002 |
| L-Arginine | 1.5 | 1.5 | 2 | 2.5 |
| Capsaicin | 0.00001 | — | — | 0.00001 |
| trans-pellitorin | — | 0.0001 | — | — |
| Nonivamide | — | — | 0.00001 | 0.00001 |
| Yogurt | ad 100 | | | |
| (0.1% fat, 3% protein) | | | | |

### Application Example 2: Dry-Based Protein Mix Drinks

The ingredients are mixed according to the recipes below and packed under protective gas or in a vacuum bag. For use, the powder is stirred in 100 ml of water (room temperature) and then consumed directly.

| Ingredient | Use in grams | | | | | | |
|---|---|---|---|---|---|---|---|
| Preparation | A | B | C | D | E | F | G |
| Sugar, fine | 5 | 5 | 5 | 5 | 5 | 5 | 5 |
| Nonivamide, | 0.03 | 0.015 | 0.03 | 0.015 | 0.03 | 0.03 | 0.015 |
| 0.1% in alcohol | | | | | | | |
| trans-pellitorin, | — | 0.015 | — | 0.015 | — | — | 0.01 |
| 1% in 1,2- | | | | | | | |
| propylene glycol/ | | | | | | | |
| diethyl malonate | | | | | | | |
| L-Arginine | 5 | 10 | 5 | 2 | 10 | 15 | 5 |
| Aroma type | 0.2 | 0.2 | 0.2 | 0.2 | — | — | — |
| cappuccino, spray | | | | | | | |
| dried | | | | | | | |
| Aroma type | — | — | — | — | 0.1 | 0.1 | 0.1 |
| strawberry, spray | | | | | | | |
| dried | | | | | | | |
| Beet color | — | — | — | — | 0.1 | 0.08 | 0.1 |
| powder, spray | | | | | | | |
| dried | | | | | | | |
| Sugar Couleur | 0.15 | 0.15 | 0.15 | 0.15 | — | — | — |
| Xanthan gum | 0.1 | 0.1 | 0.1 | 0.1 | 0.1 | 0.1 | 0.1 |
| Skimmed milk | 10 | — | — | — | 10 | — | — |
| powder | | | | | | | |
| Lactose-reduced | — | — | — | — | — | 10 | — |
| milk powder | | | | | | | |
| (Omira, | | | | | | | |
| Ravensburg) | | | | | | | |
| Lupine protein | — | — | — | 5 | — | — | — |
| powder | | | | | | | |
| Soy protein | — | 10 | — | — | — | — | — |
| hydrolysate (60% | | | | | | | |
| degree of | | | | | | | |
| hydrolysis) | | | | | | | |
| Wheat Protein- | — | — | — | — | — | — | 10 |
| hydrolysate | | | | | | | |
| (80% degree of | | | | | | | |
| hydrolysis) | | | | | | | |
| Rapeseed | — | — | 10 | — | — | — | — |
| proteininn | | | | | | | |
| (partially | | | | | | | |
| hydrolyzed, | | | | | | | |
| napin-rich) | | | | | | | |

### Application Example 3: Whey Protein Drink

Production of a fruit-whey protein drink according to the recipes mentioned below with subsequent homogenization and pasteurization.

Use in % by weight

| Ingredient | A | B |
|---|---|---|
| Whey protein isolate (min. 88% protein, | 8 | 8 |
| based on dry substance) | | |
| Fruit juice concentrate mix | 10 | 10 |
| (mango, banana, carrot, orange) | | |
| Citric acid 50% | 0..2 | 0.2 |
| Pectin | 0.4 | 0.4 |
| Vitamin mixture | 0.01 | 0.01 |
| Mineral salt mixture | 1 | 1 |
| Nonivamide | 0.0003 | 0.00015 |
| L-Arginine | 2 | 3 |
| Mango Aroma (Symrise) | 0.05 | 0.05 |
| Drinking water | ad 100 | ad 100 |

### Application Example 4; Protein Drink

The cereals and protein isolate are mixed. A syrup is cooked from the remaining ingredients (without flavoring). At the end of the cooking time, the flavoring is added. The syrup is mixed with the cereals and formed into bars.

| | Use in % by weight | | |
|---|---|---|---|
| Ingredient | A | B | C |
| Oatmeal | 13 | 13 | 13 |
| Rice Crispies | 14 | 14 | 14 |
| Corn Flakes | 10 | 10 | 10 |
| Whey Protein Isolate | 10 | — | 10 |
| (min. 88% protein, | | | |
| based on dry substance) | | | |
| Pea Protein Isolate | — | 10 | — |
| (min. 80% protein, | | | |
| based on dry substance, | | | |
| 10% degree of hydrolysis) | | | |
| Glucose syrup | 0.1 | 0.1 | 0.1 |
| Saccharose | 12 | 12 | 12 |
| Glycerin | 1 | 1 | 1 |
| Salt | 0.2 | 0.2 | 0.2 |
| Drinking water | 13.5 | 13.5 | 13.5 |
| Vegetable fat | 10 | 10 | 10 |
| Lecithin | 0.2 | 0.2 | 0.2 |
| Bittermasking Aroma (Symrise) | 0.3 | 0.3 | 0.3 |
| Caramel Aroma (Symrise) | 0.1 | 0.1 | 0.1 |
| Nonivamide | 0.00025 | 0.0005 | 0.0004 |
| L-Arginine | 5 | 4 | 3 |

1.-15. (canceled)

16. A combination agent comprising:
(a) 0.1 ppm to 10 ppm of one first saturating component, wherein the one first saturating component is nonivamide;
(b) 0.1 to 20% by weight of added free arginine or its isomers or derivatives;
in each case based on the weight of the total combination agent; and
optionally
(c) one or more second saturation component(s), optionally a protein or a protein hydrolysate; and/or
(d) one or more sensory active substance(s).

17. The combination agent according to claim 16, wherein the one or more second saturation component(s) is/are protein(s) selected from the group consisting of proteins from meat, crustaceans, shellfish, mussels, molluscs, insects, eggs, milk, algae, cereals, and other proteins from edible plants.

18. The combination agent of claim 17, wherein the meat is meat from mammals, birds, reptiles, amphibians, or fish.

19. The combination agent of claim 17, wherein the one or more saturation component(s) is/are protein(s) from milk selected from the group consisting of casein and whey.

20. The combination agent of claim 17, wherein the cereals are wheat, barley, rapeseed, sunflower, rice, potatoes, corn, soybean, beans, peas, lentils, lupins, peanuts, alfalfa, or hemp.

21. The combination agent of claim 17, wherein the other proteins from edible plants comprise proteins with intrinsically high L-arginine content.

22. The combination agent according to claim 16, wherein the one or more second saturation component(s) is/are protein hydrolysate(s) selected from the group consisting of partial hydrolysate or complete hydrolysate of proteins from meat, crustaceans, shellfish, mussels, molluscs, insects, eggs, milk, algae, cereals and other proteins from edible plants.

23. The combination agent of claim 22, wherein the meat is meat from mammals, birds, reptiles, amphibians, or fish.

24. The combination agent of claim 22, wherein the one or more saturation component(s) is/are protein hydrolysate(s) from milk selected from the group consisting of casein and whey.

25. The combination agent of claim 22, wherein the cereals are wheat, barley, rapeseed, sunflower, rice, potatoes, corn, soybean, beans, peas, lentils, lupins, peanuts, alfalfa, or hemp.

26. The combination agent of claim 22, wherein the other proteins from edible plants comprise proteins with intrinsically high L-arginine content.

27. The combination agent according to claim 16, further comprising:
(e) a carrier and/or an auxiliary means.

28. The combination agent according to claim 16, comprising:
5 to 20% by weight of added free arginine; and
optionally
1 to 95% by weight of the one or more second saturation component(s); and/or
0.00001 (0.1 ppm) to 1% by weight of the one or more sensory active substance;
in each case based on the total weight of the combination agent.

29. A method of
(i) reducing appetite; and/or
(ii) providing a feeling of satiety; and/or
(iii) reducing energy intake; and/or
(iv) reducing body weight
comprising administering to a subject in need thereof a combination agent according to claim 16.

30. A method of manufacturing an orally consumable preparation or a food ingredient comprising combining the combination agent of claim 16 with an edible ingredient.

31. An orally consumable preparation or food ingredient comprising the combination agent of claim 16.

32. The orally consumable preparation of claim 31, wherein the orally consumable preparation is selected from the group consisting of bakery products, confectionery, non-alcoholic beverages, instant beverages, meat products, insects or insect products, eggs or egg products, cereal products, dairy products, products high in vegan proteins, protein fractions, partial or complete protein hydrolysates, or physico-chemically, enzymatically or fermentatively modified proteins, meat substitutes, fruit preparations, vegetable preparations, snack foods, fat- and oil-based products, other ready-to-serve meals and soups, spices, seasoning mixtures, and sweetener preparations.

33. The orally consumable preparation or food ingredient of claim 31, comprising the combination agent in an amount of from 1 to 80% by weight, based on the total weight of the orally consumable preparation.
